SEQUENCE LISTING (42 sequences)

SEQ ID NO: 1 - DNA, 1245 bp, Mouse
atggccacatccaattcttctgcctctctgcccaccctcttctgggtcaatggctctggagacagcgtgctgagcactgacggtgctgccatgcctgtccagttccttgttctgaggatcatggttgcactggcctatggacttgtaggtatcattggcttgctgggaaatttggccgtactgtgggttctaggtaactgtggtcagcgtgtgcccggcctgtcttctgatacctttgtcttcagcctggctctagcagacttggggctggcccttactctccctttctgggcaaccgagtcagcaatggacttccactggcctttcggaagtgccctctgcaaggtagtcctgaccaccaccgtcctcagcatctatgccagcaccttcctaatcacagcactgagtatcgcgcgatactgggtggtagccatggctgtgggaccaggtagtcacctctcagtcttttgggcccgtgtggtcaccctggcagtgtgggtggcagctgccctggtgactgtgcccacagcaatctttggggctgaagttgagttgtggggcgtgtgcctctgtcttctgcgtttccccagcagatactggctgggagcttaccagctacagagggtagttctggccttcatcgtgcccttgggagtcattaccaccagttacctgctgctgttggcctttctagagcggcagcaaagatgcaggccacgacaatggcaggacagccgagtggtagcccgctctgtccgtgtcctggtggcttccttcgccctctgctgggttcccaaccatgtagtcactctctgggaaattctggtaaggtttgacctggtgccctgggacagtactttctacacctttcatacttacatccttcccatcaccacctgcttggcccacagcaacagctgcctcaaccctgtgatctattgtctcctgcggcgggagccccagcaggttcttgtcagctccttcagagctctctggtcaagactgtggcctcaaaggaaggcctgcatggaacaaatggccctcaaggaggtaggcgggagaacggtagccagcacccaggagagtggctcttctaggacacacacaaacacaatggaacacctggatgaaggatgcagcctgaacactctcctttctgagacctatcaggggcagagcccacagattctagggaggagcagctgctctctcagtcaggctgctgtgtccccaggagaagtctga

SEQ ID NO: 2 - protein, 414 aa, Mouse
MATSNSSASLPTLFWVNGSGDSVLSTDGAAMPVQFLVLRIMVALAYGLVGIIGLLGNLAVLWVLGNCGQRVPGLSSDTFVFSLALADLGLALTLPFWATESAMDFHWPFGSALCKVVLTTTVLSIYASTFLITALSIARYWVVAMAVGPGSHLSVFWARVVTLAVWVAAALVTVPTAIFGAEVELWGVCLCLLRFPSRYWLGAYQLQRVVLAFIVPLGVITTSYLLLLAFLERQQRCRPRQWQDSRVVARSVRVLVASFALCWVPNHVVTLWEILVRFDLVPWDSTFYTFHTYILPITTCLAHSNSCLNPVIYCLLRREPQQVLVSSFRALWSRLWPQRKACMEQMALKEVGGRTVASTQESGSSRTHTNTMEHLDEGCSLNTLLSETYQGQSPQILGRSSCSLSQAAVSPGEV

SEQ ID NO: 3 - DNA, 1125 bp, Homo sapiens
atgcccacactcaatacttctgcctctccacccacattcttctgggccaatgcctccggaggcagtgtgctgagtgctgatgatgctccgatgcctgtcaaattcctagccctgaggctcatggttgccctggcctatgggcttgtgggggccattggcttgctgggaaatttggcggtgctgtgggtactgagtaactgtgcccggagagcccctggcccaccttcagacaccttcgtcttcaacctggctctggcggacctgggactggcactcactctccccttttgggcagccgagtcggcactggactttcactggcccttcggaggtgccctctgcaagatggttctgacggccactgtcctcaacgtctatgccagcatcttcctcatcacagcgctgagcgttgctcgctactgggtggtggccatggctgcggggccaggcacccacctctcactcttctgggcccgaatagccaccctggcagtgtgggcggcggctgccctggtgacggtgcccacagctgtcttcggggtggagggtgaggtgtgtggtgtgcgcctttgcctgctgcgtttccccagcaggtactggctgggggcctaccagctgcagagggtggtgctggctttcatggtgcccttgggcgtcatcaccaccagctacctgctgctgctggccttcctgcagcggcggcaacggcggcggcaggacagcagggtcgtggcccgctctgtccgcatcctggtggcttccttcttcctctgctggtttcccaaccatgtggtcactctctggggtgtcctggtgaagtttgacctggtgccctggaacagtactttctatactatccagacgtatgtcttccctgtcactacttgcttggcacacagcaatagctgcctcaaccctgtgctgtactgtctcctgaggcgggagccccggcaggctctggcaggcaccttcagggatctgcggtcgaggctgtggccccagggcggaggctgggtgcaacaggtggccctaaagcaggtaggcaggcggtgggtcgcaagcaacccccgggagagccgcccttctaccctgctcaccaacctggacagagggacacccgggtga

SEQ ID NO: 4 - protein, 374 aa, Homo sapiens
MPTLNTSASPPTFFWANASGGSVLSADDAPMPVKFLALRLMVALAYGLVGAIGLLGNLAVLWVLSNCARRAPGPPSDTFVFNLALADLGLALTLPFWAAESALDFHWPFGGALCKMVLTATVLNVYASIFLITALSVARYWVVAMAAGPGTHLSLFWARIATLAVWAAAALVTVPTAVFGVEGEVCGVRLCLLRFPSRYWLGAYQLQRVVLAFMVPLGVITTSYLLLLAFLQRRQRRRQDSRVVARSVRILVASFFLCWFPNHVVTLWGVLVKFDLVPWNSTFYTIQTYVFPVTTCLAHSNSCLNPVLYCLLRREPRQALAGTFRDLRSRLWPQGGGWVQQVALKQVGRRWVASNPRESRPSTLLTNLDRGTPG

SEQ ID NO: 5 - protein, 45 aa, Homo sapiens
KESVRLCGLEYIRTVIYICASSRWQDLQTLCCTDGCSMTDLSALC

SEQ ID NO: 6 - protein, 51 aa, Homo sapiens
RAAPYGVRLCGREFIRAVIFTCGGSRWDVLAGLSSSCCKWGCSKSEISSLC

SEQ ID NO: 7 - protein, 373 aa, Monkey
MPTLNTSASPPTFWANASGGSVLSADDAPMPVKFLALRLMVALAYGLVGAVGLLGNLAVLWVLSNCARRAPGPPSDTFVFNLALADLGLALTLPFWAAESALDFHWPFGGALCKMVLTATVLNVYASIFLITALSVARYWVVAMAAGPGTHLSLFWARIATLAVWAAAALVTVPTAVFGVEGEVCGVRLCLLRFPSRYWLGAYQLQRVVLAFMVPLGVITTSYLLLLAFLQQRQRRQQDSRVVARSVRILVASFFLCWFPNHVVTLWGVLVKFDLVPWNSTFYTIQTYVLPVTTCLAHSNSCLNPVLYCLLRREPRQALADTFRDLRSRLWPQGGGWVQQVALKQVGRRWVASNPPESRPSTLLTNLDGGTPG

SEQ ID NO: 8 - protein, 373 aa, Cow
MPTPNTSAPLPAFWVNASGGSVLSAADATMPVGFLALRVSVALAYGLVGAVGLLGNSAVLWVLGNCAQRAPCPPSDTFVFNLALADLGLALTLPFWAAESALDFHWPFGGALCKMVLTATVLNIYASIFLITALSVARYWVVAMAAGPGTHLSLFWARVATLAMWVAASLVTVPTAVFGAEGEVSGVRLCLLRFPSRYWLGAYQLQRVVLAFMVPLSIITTSYLLLLAFLRRRRRRWRDSRGVAHSIRILLASFFLCWFPNHVVTLWGVLVKFDLVPWDSTFYTVHTYVFPVTTCLAHTNSCLNPVLYCLLRQEPRQALADTFRDLRARLWPQGRGWVEQVALKEMGRRWTESTPQEGGLSTMLTNLDKGNPG

SEQ ID NO: 9 - protein, 373 aa, Pig
MPTPNTSAPLPAFWVNASGGSVLSADDATMPVGFLALRVMVALAYGLVGAVGLLGNLAVLWVLGNCARRAPCPPSDTFVFNLALADLGLALTLPFWAAESALDFHWPFGGALCKMILTATVLNIYASIFLITALSVARYWVVAMAGGPGTHLSLFWARVATLAVWVAAALVTVPTAVFGAEGELCGVRLCLLRFPSRYWLGAYQLQRVVLAFMVPLGIITTSYLLLLAFLRRRRQRRQDNRVVARSIRILLASFFLCWFPNHVVTFWGVLVKFDLVPWDSTFYTIHTYVFPVTTCLAHSNSCLNPVLYCLLRREPRRALEVTFRDLRARLWPQGRGWVEQVALKEVGRRWEEITPRDGGPSAMPTNRDKGTAG

SEQ ID NO: 10 - DNA, 40 bp, Artificial
  Human GPCR142 forward PCR primer
actggaattcgccaccatgcccacactcaatacttctgcc

SEQ ID NO: 11 - DNA, 40 bp, Artificial
  Human GPCR142 reverse PCR primer
actgacgcggccgctcacccgggtgtccctctgtccaggt

SEQ ID NO: 12 - DNA, 24 bp, Artificial
  Human GPCR142 5′-UTR primer for PCR of monkey GPCR142
aggtggtgggttgtcctttccaca

SEQ ID NO: 13 - DNA, 22 bp, Artificial
  Human GPCR142 3′-UTR primer for PCR of monkey GPCR142
ctcaaggatcctacacttggtg

SEQ ID NO: 14 - DNA, 44 bp, Artificial
  Mouse GPCR142 forward PCR primer
acgatagaattcgccaccatggccacatccaattcttctgcctc

SEQ ID NO: 15 - DNA, 40 bp, Artificial
  Mouse GPCR142 reverse PCR primer
acgatagcggccgctcagacttctcctggggacacagcag

SEQ ID NO: 16 - DNA, 41 bp, Artificial
  Bovine GPCR142 forward PCR primer
actagagaattcgccaccatgcccacgcccaacacctctgc

SEQ ID NO: 17 - DNA, 41 bp, Artificial
  Bovine GPCR142 reverse PCR primer
actagagcggccgcccagaaagaggagggggtttaacttgc

SEQ ID NO: 18 - DNA, 41 bp, Artificial
  Porcine GPCR142 forward PCR primer
atgatagaattcgccaccatgcccacacccaatacctctgc

SEQ ID NO: 19 - DNA, 40 bp, Artificial
  Porcine GPCR142 reverse PCR primer
actagagcggccgcccagaaagagggggattagcttgctc

SEQ ID NO: 20 - DNA, 45 bp, Artificial
  Human Relaxin3 forward PCR primer
acgatcgtcgacgccaccatggccaggtacatgctgctgctgctc

SEQ ID NO: 21 - DNA, 41 bp, Artificial
  Human Relaxin3 reverse PCR primer
acgataaagcttctagcaaaggctactgatttcacttttgc

SEQ ID NO: 22 - DNA, 52 bp, Artificial
  Human Relaxin3 propeptide coding sequence, forward PCR primer
acgatagaattcgatgacgacgataagcgggcagcgccttacggggtcaggc

SEQ ID NO: 23 - DNA, 44 bp, Artificial
  Human Relaxin3 propeptide coding sequence, reverse PCR primer
actataggatccctagcaaaggctactgatttcacttttgctac

SEQ ID NO: 24 - DNA, 40 bp, Artificial
  Human Relaxin3 propeptide/Furin site 5-prime end forward PCR primer
acgatactgcaggccgccatgctgaccgcagcgttgctga

SEQ ID NO: 25 - DNA, 45 bp, Artificial
  Human Relaxin3 propeptide/Furin site 5-prime end reverse PCR primer
cagccaggacatctcgtcggccccgaagaaccccaggggttcctt

SEQ ID NO: 26 - DNA, 46 bp, Artificial
  Human Relaxin3/Furin site 3-prime end forward PCR primer
ggttcttcggggccgacgagatgtcctggctggcctttccagcagc

SEQ ID NO: 27 - DNA, 44 bp, Artificial; identical to SEQ ID NO: 23
  Human Relaxin3/Furin site 3-prime end reverse PCR primer

SEQ ID NO: 28 - DNA, 44 bp, Artificial
  Human Furin forward PCR primer
gactagaagcttgccaccatggagctgaggccctggttgctatg

SEQ ID NO: 29 - DNA, 39 bp, Artificial
  Human Furin reverse PCR primer
gacgatagcggccgcagtgggctcatcagagggcgctct

SEQ ID NO: 30 - DNA, 52 bp, Artificial
  Human INSL5/Furin site 5-prime end forward PCR primer
atataggaattcgacgacgacgacaagaaggagtctgtgagactctgtgggc

SEQ ID NO: 31 - DNA, 65 bp, Artificial
  Human INSL5/Furin site 5-prime end reverse PCR primer
gtgcaacacaaagtttgtaaatcttgtcttcgccaacgtgaatgcttctttgacttccaaagctc

SEQ ID NO: 32 - DNA, 52 bp, Artificial; identical to SEQ ID NO: 30
  Human INSL5/Furin site 3-prime end forward PCR primer

SEQ ID NO: 33 - DNA, 80 bp, Artificial
  Human INSL5/Furin site 3-prime end reverse PCR primer
actagaaagcttttagcaaagagcactcaaatcagtcatggaacagccatcagtgcaacacaaagtttgtaaatcttgtc

SEQ ID NO: 34 - DNA, 25 bp, Artificial
  Human GPCR142 mRNA probe, forward PCR primer
ttcactggcccttcggaggtgccct

SEQ ID NO: 35 - DNA, 25 bp, Artificial
  Human GPCR142 mRNA probe, reverse PCR primer
cagagagtgaccacatggttgggaa

SEQ ID NO: 36 - DNA, 30 bp, Artificial
  Human beta-actin mRNA probe, forward PCR primer
atatcgccgcgctcgtcgtcgacaacggct

SEQ ID NO: 37 - DNA, 30 bp, Artificial
  Human beta-actin mRNA probe, reverse PCR primer
tttgcggtggacgatggaggggccggactc

SEQ ID NO: 38 - protein, 469 aa, Homo sapiens
MQMADAATIATMNKAAGGDKLAELFSLVPDLLEAANTSGNASLQLPDLWWELGLELPDGAPPGHPPGSGGAESADTEARVRILISVVYWVVCALGLAGNLLVLYLMKSMQGWRKSSINLFVTNLALTDFQFVLTLPFWAVENALDFKWPFGKAMCKIVSMVTSMNMYASVFFLTAMSVTRYHSVASALKSHRTRGHGRGDCCGRSLGDSCCFSAKALCVWIWALAALASLPSAIFSTTVKVMGEELCLVRFPDKLLGRDRQFWLGLYHSQKVLLGFVLPLGIIILCYLLLVRFIADRRAAGTKGGAAVAGGRPTGASARRLSKVTKSVTIVVLSFFLCWLPNQALTTWSILIKFNAVPFSQEYFLCQVYAFPVSVCLAHSNSCLNPVLYCLVRREFRKALKSLLWRIASPSITSMRPFTATTKPEHEDQGLQAPAPPHAAAEPDLLYYPPGVVVYSGGRYDLLPSSSAY

SEQ ID NO: 39 - DNA, 43 bp, Artificial
  Monkey GPCR142 forward PCR primer
actagagaattcgccaccatgcccacactcaatacttctgcct

SEQ ID NO: 40 - DNA, 40 bp, Artificial
  Monkey GPCR142 reverse PCR primer
actagagcggccgcttacccgggtgtccctccgtccaggt

SEQ ID NO: 41 - DNA, 21 bp, Artificial
  Human GPCR142 forward primer for PCR of bovine and porcine GPCR142
accaatctctgatgccctgcg

SEQ ID NO: 42 - DNA, 24 bp, Artificial
  Human GPCR primer reverse primer for PCR of bovine and porcine GPCR142
gagttggggatcaaagatcagact

(19) United States
(12) Patent Application Publication
(10) Pub. No.: US 2005/0059089 A1
(43) Pub. Date: Mar. 17, 2005

(54) Complexes of GPCR142 and relaxin3 or INSL5, and their production and use

(76) Inventors: Chester Kuei, Murrieta, CA (US); Changlu Liu, San Diego, CA (US); Timothy W. Lovenberg, San Diego, CA (US); Steven W. Sutton, Carlsbad, CA (US); Jingcai Chen, Beijing (CN)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/913,713

(22) Filed: Aug. 6, 2004

Related U.S. Application Data
(60) Provisional application No. 60/493,941, filed on Aug. 7, 2003.
(60) Provisional application No. 60/580,083, filed on Jun. 16, 2004.

(51) Int. Cl. C12Q 1/68; G01N 33/53; C07K 14/705

(52) U.S. Cl. 435/7.1; 530/350

(57) ABSTRACT

A GPCR142/relaxin3 and GPCR142/INSL5 complexes are described. The complexes are useful as assay reagents for screening for modulators of GPCR142 receptor activity.

### CROSS-REFERENCES TO RELATED APPLICATIONS

This application claims priority benefit to U.S. Provisional Application No. 60/493,941, filed 7 Aug. 2003, entitled “Relaxin3-GPR142 Complexes And Their Production And Use”. This application also claims priority benefit to U.S. Provisional Application No. 60/580,083, filed 16 Jun. 2004, entitled “GPCR142-INSL5 Complexes And Their Production And Use”. The disclosures of these priority applications are incorporated by reference herein.

### FIELD OF THE INVENTION

The present invention relates to GPCR142/relaxin3 complexes and GPCR142/INSL5 complexes. The invention also relates to processes of preparing such complexes and methods of their use. The invention further relates to GPCR142 peptides from distinct species.

### BACKGROUND OF THE INVENTION

As described in International Application No. PCT/US2004/005666, filed 25 Feb. 2004, relaxin3 is a natural ligand for GPCR135, a G-protein coupled receptor (GPCR). Having discovered that GPCR135 is the endogenous receptor for relaxin3, which is also referred to as INSL7 (Genbank Accession No.: NM_{—}173184), a DNA sequence contig (Genbank Accession No.: AL355388) containing a gene with 40% homology to GPCR135 at the amino acid level was discovered, which is named GPCR142.

GPCRs are transmembrane receptor proteins that are responsible for the transduction of a diverse array of extracellular signals, including hormones, neurotransmitters, peptides, lipids, ions, light, odorants, nucleotides, fatty acid derivatives, and other chemical mediators. See, e.g., WIPO Publication No. WO 02/00719. GPCRs are of particular importance to drug discovery because they have been established as excellent drug targets: they are the targets of 50% of marketed drugs. An increasing number of diseases have been found to be associated with GPCRs. Drugs targeting GPCRs have been used to treat a wide range of disorders from cardiovascular to gastro-intestinal to CNS and others (Wilson et al., 1998, British J. of Pharmacology 125:1387-1392).

The GPCR-mediated signal transduction event is often initiated upon binding of a specific ligand to the GPCR. Each GPCR is composed of an extracellular N-terminal domain, seven distinct transmembrane segments, and an intracellular C-terminal domain. Binding of the ligand to an extracellular N-terminal domain, transmembrane domain, or intracellular loop of a GPCR results in a conformational change that leads to activation of intracellular heterotrimeric GTP-binding proteins (G proteins) associated with the GPCR. These activated G proteins in turn mediate a variety of intracellular responses that regulate cell physiology. Therefore, the ligand provides means of elucidating the physiological function of the GPCR as well as methods of screening for compounds that regulate the signal transduction activity of the GPCR.

At present, only about 200 GPCRs are classified as known GPCRs that are activated by around 70 known ligands. Through sequence analyses, it was discovered that GPCRs belong to one of the largest superfamilies of the human genome: evaluated at over 1000 genes encoding GPCRs (Civelli et al., 2001, Trends in Neurosciences 24:230-237). A large number of putative GPCRs are described as orphan receptors because their natural ligands are unknown. Some of these uncharacterized orphan GPCRs may be useful as therapeutic targets. The identification of the specific ligand to a GPCR is the key to harnessing the potential therapeutic benefits of these orphan GPCRs (Howard et al., 2001, Trends in Pharmacological Sciences 22:132-140).

As noted above, relaxin3 (also known as INSL7) has been found to be a ligand for GPCR135 as well as for GPCR142. See Liu et al., 2003a, “Identification of relaxin-3 as an endogenous ligand for the orphan G-protein coupled receptor GPCR135,” JBC 278:50754-50764; and Liu et al. (2003b), “Identification of a second relaxin-3 receptor, GPCR142,” JBC 278:50765-50770. Relaxin3 is a member of the insulin/relaxin superfamily. Members in this family are characterized by peptide subunits (A-chain and B-chain) linked by three disulfide bonds. Two of the three disulfide bonds are inter-subunit bonds and another one is an intra-chain bond in the B-chain. In the family, insulin, IGF1, and IGF2 have been reported to be involved in the regulation of glucose metabolism and signal through tyrosine kinase/growth factor receptors, which are single transmembrane receptors. Two other members in the family are relaxin and INSL3, ligands for LGR7 and/or LGR8, which are GPCRs with leucine-rich repeats at the N-terminal extra-cellular domain. Relaxin3 was also reported to be an additional ligand for LGR7. Another member of the relaxin/insulin superfamily is INSL5 (Conklin et al. 1999, Genomics 60(1):50-56).

Having discovered that GPCR135 is the endogenous receptor for relaxin3, which is also referred to as INSL7 (Genbank Accession No.: NM_{—}173184), we found a DNA sequence contig (Genbank Accession No.: AL355388) containing a gene with 40% homology to GPCR135 at the amino acid level, which we named GPCR142. However, a ligand for GPCR142 had not been previously identified.

### SUMMARY OF THE INVENTION

It has now been discovered that GPCR142 is selectively activated by relaxin3; furthermore, it has now been discovered that GPCR142, but not GPCR135, is selectively activated by INSL5.

In one general aspect, the invention therefore relates to a receptor-ligand complex comprising a receptor component containing GPCR142 or an active fragment of GPCR142 bound to a ligand component selected from relaxin3 or an active fragment of relaxin3, or INSL5 or an active fragment of INSL5, wherein at least one of the receptor and ligand components is in a substantially pure form.

In other general aspects, the invention relates to: an isolated polynucleotide having a nucleotide sequence corresponding to SEQ ID NO: 1 or a complement thereof; an isolated polypeptide having an amino acid sequence corresponding to SEQ ID NO: 2, 4, 7, 8, and 9; a vector consisting of a polynucleotide encoding such polypeptide; and a recombinant host cell comprising such vector.

Further general aspects of the invention relate to assay or screening methods. For example, one general method of identifying a compound that increases or decreases a biological activity of either a GPCR142/relaxin3 complex or a GPCR142/INSL5 complex comprises: contacting a test sample comprising a compound and a buffering solution with an assay reagent comprising a receptor-ligand complex of the invention; determining the biological activity of the receptor-ligand complex; and comparing the measurement obtained in that determination with a control measurement wherein the receptor-ligand complex has been contacted with the buffering solution. Another general assay method of identifying a compound that binds to GPCR142 or an active fragment thereof comprises: contacting GPCR142 or an active fragment thereof with a test compound and with either a labeled relaxin3 or an active fragment thereof, or a labeled INSL5 or an active fragment thereof; and determining the amount of the labeled ligand component or active fragment thereof that binds to the GPCR142 or active fragment thereof, and then comparing that amount with a control measurement wherein the GPCR142 or active fragment thereof has been contacted with the labeled ligand component or active fragment thereof in the absence of test compound. A further general assay relates to a method for identifying a compound that binds GPCR142 and mimics either relaxin3 or INSL5, comprising: contacting a test compound with an assay reagent comprising GPCR142 or an active fragment thereof; determining a biological activity of the GPCR142 or active fragment thereof; and comparing this result with that of a control measurement wherein the GPCR142 or an active fragment thereof was contacted with either relaxin3 or an active fragment thereof or INSL5 or an active fragment thereof, in the absence of the test compound.

Other aspects, features and advantages of the invention will be apparent from the following disclosure, including the detailed description of the invention and its preferred embodiments and the appended claims.

### BRIEF DESCRIPTION OF THE DRAWINGS

FIG. 1 provides an amino acid sequence comparison between human GPCR142 (SEQ ID NO:2) and GPCR135 (SEQ ID NO:38). Putative transmembrane regions TM1 to TM7 are underlined.

FIGS. 2A-2C show the characterization of GPCR142 by radioligand binding assays.

FIG. 2A illustrates the binding of relaxin3 to membranes from COS-7 cells expressing GPCR142. ¹²⁵I-relaxin3 was used as the radioligand at 100 pM as the final concentration. Membranes from COS-7 cells expressing GPCR142 were incubated with ¹²⁵I-relaxin3 (SEQ ID NO: 6) and then filtered though GFC filters. The bound ¹²⁵I-relaxin3 was counted in a TopCount NTX. The non-specific binding controls were performed at the same conditions in the presence of 5 μM of unlabeled relaxin3. Mock transfected COS-7 cells were used as the negative controls in a parallel experiment under the same conditions.

FIG. 2B depicts the results of saturation binding assays for GPCR142. Membranes from COS-7 cells expressing GPCR142 were incubated with different concentrations of ¹²⁵I-relaxin3 in the binding assays. The non-specific binding controls were obtained by performing the binding experiments at the same conditions in the presence of 5 μM of unlabeled relaxin3. Specific binding was calculated by subtracting the non-specific binding from the total binding.

FIG. 2C shows the results of competition binding assays for GPCR142. In the binding assays, membranes from COS-7 cells expressing GPCR142 were incubated with 100 μM of ¹²⁵I-relaxin3 in the presence of various concentrations of different competitors.

FIG. 3 shows relaxin3 stimulation of ³⁵S-GTPγS binding in GPCR142-expressing cells. Different peptides with various concentrations were added to the human GPCR142 expressing cell membranes to stimulate GTPγS incorporation. The specific ³⁵S-GTPγS incorporation was obtained by subtracting counts without ligand from the counts with ligand.

FIG. 4 illustrates the inhibition of forskolin-stimulated cAMP production by relaxin3. Cells stably expressing GPCR142 were established in SK-N-MC cells harboring a β-galactosidase gene under the control of a CRE promoter (SK-N-MC/CRE-β-gal). Different peptides with various concentrations were added to the cells. Forskolin was then added to all samples at a final concentration of 5 μM. β-Gal activities in the samples, which reflect the cAMP concentration in the cells, were measured using a β-gal activity assay.

FIG. 5 depicts the relaxin3 stimulation of Ca²⁺ mobilization in HEK293 cells co-expressing GPCR142 and G_{α16}. HEK293 cells, either transfected with G_{α16} alone (G_{α16}), or co-transfected by human GPCR142 and G_{α16} (GPCR142/G_{α16}), were used for Ca²⁺ mobilization assays using different concentrations of relaxin3 as the ligand. Ligand stimulated intracellular Ca²⁺ mobilization was monitored by FLIPR. HEK293 cells co-transfected by G_{α16} and GPCR135 (GPCR135/G_{α16}) were used in the same experiment for comparison.

FIG. 6 shows RT-PCR detection of GPCR142 mRNA expression profiles in different human tissues. The PCR products were run in 2% agarose gels, stained with ethidium bromide, and visualized under UV irradiation.

FIG. 7 provides images of brain sections showing expression of GPCR142 mRNA by in situ hybridization. Sections are pseudocolor images from autoradiograms processed on the Fujifilm Bio-Imaging Analyzer System. Sagittal brain sections were hybridized with GPCR142 probe using antisense probe (A) and sense probe (B). Coronal brain sections show light distribution of GPCR142 mRNA in the septal region (C) and thalamus region (D).

FIG. 8 is a sequence alignment illustrating the homology between the polypeptide sequences of human INSL5 (SEQ ID NO.:5) and human relaxin3 (SEQ ID NO.:6).

FIG. 9 provides amino acid sequence comparisons of human GPCR142 (SEQ ID NO.:4), monkey GPCR142 (SEQ ID NO.:7), bovine GPCR142 (SEQ ID NO.:8), porcine GPCR142 (SEQ ID NO.:9), and mouse GPCR142 (SEQ ID NO.:2). The consensus sequence is indicated in bold. The putative seven transmembrane domains are underlined and denoted TM1 to TM7. The DNA sequences for mouse, monkey, bovine (cow), and porcine (pig) GPCR142 were deposited in GenBank (Accession Nos.: AY633765; AY633766; AY633767; AY633768, respectively).

FIG. 10 provides the nucleotide sequence for human GPCR142 DNA (SEQ ID NO: 3).

FIG. 11 provides the nucleotide sequence for mouse GPCR142 DNA (SEQ ID NO: 1).

FIG. 12 is a plot of the results of GTPγS binding assays showing that human recombinant INSL5 stimulates GTPγS binding in GPCR142 expression membranes, but not in GPCR135 membranes.

FIG. 13 is a plot of the comparative results from β-galactosidase assays with each of INSL5 and relaxin3 for both GPCR135 and GPCR142.

FIG. 14 is a plot of the comparative results from Ca mobilization assays with each of INSL5 and relaxin3 for both GPCR135 and GPCR142.

FIG. 15 depicts comparative results from competition binding assays using ¹²⁵I-relaxin3 as the tracer with each of INSL5 and relaxin3 as competitors for both GPCR135 and GPCR142.

### DETAILED DESCRIPTION OF INVENTION AND ITS PREFERRED EMBODIMENTS

The disclosures of all publications cited below are hereby incorporated by reference herein. Unless defined otherwise, all technical and scientific terms used herein have the same meaning as commonly understood to one of ordinary skill in the art to which this invention pertains.

The following are abbreviations that are at times used in this specification below: bp=base pair; Ca²⁺=calcium ion; cAMP=cyclic adenosine monophosphate; cDNA=complementary DNA; CNS=central nervous system; kb=kilobase; 1000 base pairs; kDa=kilodalton; GPCR=G-protein coupled receptor; G protein=GTP-binding protein; GTP=guanosine 5′-triphosphate; nt=nucleotide; PAGE=polyacrylamide gel electrophoresis; PCR=polymerase chain reaction; SDS=sodium dodecyl sulfate; SiRNA=small interfering RNA; and UTR=untranslated region.

The terms “including,” “comprising” and “containing” are used herein in their open, non-limiting sense.

“An activity”, “a biological activity”, or “a functional activity” of a polypeptide or nucleic acid refers to an activity exerted by a polypeptide or nucleic acid molecule as determined in vivo or in vitro according to standard techniques. Such activities can be a direct activity, such as an association with or an enzymatic activity on a second protein, or an indirect activity, such as a cellular signaling activity mediated by interaction of the protein with a second protein. For example, an illustrative biological activity of relaxin3 is its ability to bind GPCR135 or GPCR142 and initiate the signal transduction event(s) conducted thereby. An exemplary biological activity of GPCR142 is that, like GPCR135, upon binding to relaxin3, it activates a chain of events that alters the concentration of intracellular signaling molecules (second messenger molecule), such as cyclic AMP and calcium via activating G-protein, which has a high affinity to GTP. These intracellular signaling molecules in turn alter the physiology and behavior of the cell.

A “clone” is a population of cells derived from a single cell or common ancestor by mitosis. A “cell line” is a clone of a primary cell that is capable of stable growth in vitro for many generations.

A “disorder related to the relaxin3 and GPCR142 complex” means a disorder or disease associated with overactivity or insufficient activity of the relaxin3 and GPCR142 complex, and conditions that accompany such a disorder or disease. The term “overactivity” refers to either increased expression of the ligand and receptor complex, or increased biological activities of the complex. The term “insufficient activity” refers to either decreased expression of the ligand and receptor complex, or decreased biological activities of the complex.

“Genetic variant” or “variant” means a specific genetic variant which is present at a particular genetic locus in at least one individual in a population and that differs from the wild type.

As used herein, a “GPCR142” refers to a polypeptide that corresponds to human GPCR142 (SEQ ID NO:4), mouse GPCR142 (SEQ ID NO:2), monkey GPCR142(SEQ ID NO:7), bovine GPCR142 (SEQ ID NO:8), or porcine GPCR142(SEQ ID NO:9), or is capable of binding to antibodies, e.g., polyclonal or monoclonal antibodies, raised against a human, mouse, monkey, bovine, or porcine GPCR142 protein described herein. The cDNA or protein sequence of human GPCR142 is provided in U.S. Pat. No. 5,955,309 and WIPO Publication Nos. WO 01/36471, WO 02/61087, and WO 02/00719, the disclosures of which are incorporated by reference herein.

As used herein, the term “host cell” refers to a cell that contains a DNA molecule either on a vector or integrated into a cell chromosome. A host cell can be either a native host cell that contains the DNA molecule endogenously or a recombinant host cell.

An “isolated” nucleic acid molecule is one that is substantially separated from nucleic acid molecules with differing nucleic acid sequences. Embodiments of the isolated nucleic acid molecule of the invention include cDNA and genomic DNA and RNA, preferably of human origin.

An “isolated” or “purified” protein or biologically active fragment or portion thereof is substantially free of cellular material or other contaminating proteins from the cell or tissue source from which the protein is produced and isolated, or substantially free of chemical precursors or other chemicals when the protein is chemically synthesized. For example, protein that is substantially free of cellular material can include preparations of protein having less than about 30%, or preferably 20%, or more preferably 10%, or even more preferably 5% (by dry weight) of contaminating proteins. When the protein or biologically active portion thereof is recombinantly produced, it is also preferably substantially free of culture medium, e.g., culture medium representing less than about 20%, or more preferably 10%, or even more preferably 5% of the volume of the protein preparation. When the protein is produced by chemical synthesis, it is preferably substantially free of chemical precursors or other chemicals, i.e., it is separated from chemical precursors or other chemicals that are involved in the synthesis of the protein. Accordingly such preparations of the protein have less than about 30%, or preferably 20%, or more preferably 10%, or even more preferably 5% (by dry weight) of chemical precursors or compounds other than the polypeptide of interest.

Isolated biologically active polypeptides can have several different physical forms. The isolated polypeptide can exist as a full-length nascent or unprocessed polypeptide, or as partially processed polypeptides or combinations of processed polypeptides. The full-length nascent polypeptide can be post-translationally modified by specific proteolytic cleavage events that result in the formation of fragments of the full-length nascent polypeptide. A fragment, or physical association of fragments, can have the biological activity associated with the full-length polypeptide; of course, the degree of biological activity associated with individual fragments can vary.

The term “linker region” or “linker domain” refers to one or more polynucleotide or polypeptide sequences that are used in the construction of a cloning vector or fusion protein. The function of a linker region can include introduction of cloning sites into the nucleotide sequence, introduction of a flexible component or space-creating region between two protein domains, or creation of an affinity tag to facilitate a specific molecule interaction. A linker region can be introduced into a fusion protein, if desired, during polypeptide or nucleotide sequence construction.

The term “nucleic acid” as used herein refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single- or double-stranded. However, linkages may include any of the linkages known in the art, including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like. Furthermore, the term “nucleic acid sequences” contemplates the complementary sequence and includes any nucleic acid sequence that is substantially homologous to the both the nucleic acid sequence and its complement.

“Polynucleotide” refers to a linear polymer of at least 2 nucleotides joined together by phosphodiester bonds and may comprise ribonucleotides or deoxyribonucleotides.

“Polymorphism” refers to a set of genetic variants at a particular genetic locus among individuals in a population.

“Polypeptide” refers to any peptide or protein comprising two or more amino acids joined to each other in a linear chain by peptide bonds. As used herein, the term refers both to short chains, which are also referred to in the art as, e.g., peptides, oligopeptides and oligomers, and to longer chains, which are often referred to in the art as proteins, of which there are many types. It will be appreciated that polypeptides often contain amino acids other than the 20 amino acids commonly referred to as the 20 naturally occurring amino acids, and that many amino acids, including the terminal amino acids, can be modified in a given polypeptide, either by natural processes, such as processing and other post-translational modifications, and by chemical modification techniques known in the art. Common modifications that occur naturally in polypeptides are too numerous to list exhaustively here, but are described in basic texts and in more detailed monographs, as well as in research literature, and are therefore within the purview of persons of ordinary skill in the art. Among the known modifications which can be present in polypeptides of the present invention are, to name an illustrative few, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Several common modifications, such as glycosylation, lipid attachment, sulfation, gamma-carboxylation of glutamic acid residues, hydroxylation and ADP-ribosylation, are described in many basic texts, including PROTEINS—STRUCTURE AND MOLECULAR PROPERTIES, 2nd ed., T. E. Creighton, W. H. Freeman and Company, New York (1993). Many reviews are also available on this subject, such as those provided by Wold, “Posttranslational Protein Modifications: Perspectives and Prospects,” pgs. 1-12 in POSTTRANSLATIONAL COVALENT MODIFICATION OF PROTEINS, Johnson (ed.), Academic Press, New York (1983); Seifter et al. (1990), Meth. Enzymol. 182, 626-646; and Rattan et al., “Protein Synthesis: Posttranslational Modifications and Aging” (1992), Ann. N.Y. Acad. Sci. 663, 48-62.

It will be appreciated, as is known and as noted above, that polypeptides are not always entirely linear. For instance, polypeptides can be post-translationally modified, including via natural processing or through human manipulation. Circular, branched and branched-circular polypeptides can be synthesized by non-translation natural processes and by entirely synthetic methods as well. Modifications can occur anywhere in a polypeptide, including the peptide backbone, the amino acid side-chains, and the amino or carboxyl termini. For example, blockage of the amino or carboxyl group or both in a polypeptide by a covalent modification is common in naturally occurring and synthetic polypeptides, and such modifications can be present in polypeptides of the present invention. For instance, the amino terminal residue of polypeptides made in E. coli or other cells, prior to proteolytic processing, almost invariably will be N-formylmethionine. During post-translational modification of the peptide, a methionine residue at the NH₂-terminus can be deleted. Accordingly, this invention contemplates the use of both the methionine-containing and the methionineless amino terminal variants of the protein.

The modifications that occur in a polypeptide often will be a function of how it is made. For polypeptides made by expressing a cloned gene in a host, for instance, the nature and extent of the modifications in large part will be determined by the host cell posttranslational modification capacity and the modification signals present in the polypeptide amino acid sequence. For instance, as is known, glycosylation often does not occur in bacterial hosts such as, for example, E. coli. Accordingly, when glycosylation is desired, a polypeptide should be expressed in a glycosylating host, generally a eukaryotic cell. Insect cells often carry out the same posttranslational glycosylations as mammalian cells and, for this reason, insect-cell expression systems have been developed to express efficiently mammalian proteins having native patterns of glycosylation, among other things. Similar considerations apply to other modifications. It will be appreciated that the same type of modification can be present in the same or varying degree at several sites in a given polypeptide. Also, a given polypeptide can contain many types of modifications. In general, as used herein, the term “polypeptide” encompasses all such modifications, including those that are present in polypeptides synthesized recombinantly by expressing a polynucleotide in a host cell.

“Prohormone convertase (PC)” refers to a family of Ca²⁺-dependent serine proteases, all of which possess homology to the bacterial endoproteases subtilisin (bacteria) and yeast kexin. This family, also known as furin/paired basic amino-acid-cleaving enzyme (PACE), includes, e.g., PC1/PC3, PC2, PC4, PACE4, PC5/PC6, and PC7/PC8/lymphoma proprotein convertase, and SKI-1. They share a degree of amino-acid identity of 50-75% within their catalytic domains (for a review on. PCs, see Seidah et al., 1999, Brain Res, 27:848(1-2), 45-62).

“Promoter” means a regulatory sequence of DNA that is involved in the binding of RNA polymerase to initiate transcription of a gene. Promoters are often upstream (i.e., 5′ to 3′) the transcription initiation site of the gene. A “gene” is a segment of DNA involved in producing a peptide, polypeptide, or protein, including the coding region, non-coding regions preceding (“5′UTR”) and following (“3′UTR”) coding region, as well as intervening non-coding sequences (“introns”) between individual coding segments (“exons”). “Coding” refers to the specification of particular amino acids or termination signals in three-base triplets (“codons”) of DNA or mRNA.

A “recombinant host cell” is a cell that has been transformed or transfected by an exogenous DNA sequence. As used herein, a cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed or transfected cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the exogenous DNA. Recombinant host cells may be prokaryotic or eukaryotic, including bacteria such as E. coli, fungal cells such as yeast, mammalian cells such as cell lines of human, bovine, porcine, monkey and rodent origin, and insect cells such as Drosophila and silkworm derived cell lines. It is further understood that the term “recombinant host cell” refers not only to the particular subject cell, but also to the progeny or potential progeny of such a cell. Because certain modifications can occur in succeeding generations due to either mutation or environmental influences, such progeny may not, in fact, be identical to the parent cell, but are still included within the scope of the term as used herein.

A “recombinant polypeptide” refers to a polypeptide produced by recombinant DNA techniques, i.e., produced from cells transformed by an exogenous DNA construct encoding the desired polypeptide. A “synthetic polypeptide” refers to that prepared by chemical synthesis.

A “relaxin3” refers to a polypeptide that: (1) has greater than about 60% amino acid sequence identity to human relaxin3 (SEQ ID NO: 6) (Bathgate et al., 2002. J. Biol. Chem. 277: 1148-1157, GenBank Protein Accession No.: NP_{—}543140); (2) is capable of binding to antibodies, e.g., polyclonal or monoclonal antibodies, raised against a human relaxin3, protein described herein; or (3) is encoded by a polynucleotide that specifically hybridizes under stringent hybridization conditions to a nucleic acid molecule having a sequence that has greater than about 60% nucleotide sequence identity to human relaxin3 cDNA (GenBank nucleotide Accession No: NM_{—}080864).

An “INSL5” refers to a polypeptide that: (1) has greater than about 60% amino acid sequence identity to human INSL5 (SEQ ID NO: 5)(GenBank Accession No. AF133816); (2) is capable of binding to antibodies, e.g., polyclonal or monoclonal antibodies, raised against a human INSL5 protein described herein; or (3) is encoded by a polynucleotide that specifically hybridizes under stringent hybridization conditions to a nucleic acid molecule having a sequence that has greater than about 60% nucleotide sequence identity to human INSL5 CDNA.

A “ligand component” refers to either relaxin3 or an active fragment thereof, or INSL5 or an active fragment thereof.

In preferred embodiments, the ligand component is a polypeptide having greater than 65, 70, 75, 80, 85, 90, or 95 percent amino acid sequence identity either to human relaxin3 or to human INSL5. In other preferred embodiments, the ligand component is a polypeptide encoded by a polynucleotide that specifically hybridizes under stringent hybridization conditions to a nucleic acid molecule having a sequence that has greater than 65, 70, 75, 80, 85, 90, or 95 percent nucleotide sequence identity either to human relaxin3 cDNA or human INSL5 cDNA.

Exemplary relaxin3 ligands include relaxin3 orthologs that have been identified in human (GenBank protein Accession No.: NP_{—}543140), rat (Burazin et al., 2002, J. Neurochem. 82: 1553-1557; GenBank protein Accession No. NP_{—}733767; 76.4% sequence identity to that of human,), mouse (Bathgate et al., 2002. J. Biol. Chem. 277: 1148-1157; GenBank protein accession number XP_{—}146603; 78.7% sequence identity to that of human), and other animals, including pig and monkey. A “relaxin3” includes all three forms of relaxin3: the relaxin3 pre-propeptide or precursor, an intracellular polypeptide consisting of the signal sequence and the relaxin3 propeptide sequence; the relaxin3 propeptide, a secreted polypeptide having sequences for chains A, C, and B of relaxin3 linked by protease cleavage sites; and mature relaxin3 peptide, a secreted protein consisting of relaxin3 polypeptide chains A and B, linked by disulfide bridges. An “active fragment of relaxin3” includes any fragments of a relaxin3 protein that maintains a biological activity of relaxin3, such as binding to a GPCR142. The protein or cDNA sequence of human relaxin3 has been disclosed in WIPO Publication Nos. WO 01/68862, WO 01/81562, and WO 02/22802. The protein or cDNA sequence of rat or mouse relaxin3 has been disclosed in WIPO Publication No. WO 01/81562.

Exemplary INSL5 ligands include human INSL5. An “INSL5” includes all of its forms (e.g., pre-propeptide or precursor; propeptide; and mature peptide). An “active fragment of INSL5” includes any fragments of an INSL5 protein that maintain biological activity of INSL5 by binding to GPCR142.

A “second messenger response of a cell” refers to cellular response of the cell mediated through activation of a GPCR142 upon binding of an inventive ligand component. It may include, e.g., signal transduction event or a change in intracellular concentration of a second messenger molecule, such as proton (pH), calcium, or cAMP.

“Sequence” means the linear order in which monomers occur in a polymer, for example, the order of amino acids in a polypeptide or the order of nucleotides in a polynucleotide.

“Sequence identity or similarity or homology”, as known in the art, is the relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences.

To determine the percent identity or similarity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same or similar amino acid residue or nucleotide as the corresponding position in the second sequence, then the molecules are identical or similar at that position. The percent identity or similarity between the two sequences is a function of the number of identical or similar positions shared by the sequences (i.e., % identity=of identical positions/total# of positions (e.g., overlapping positions)×100).

Both identity and similarity can be readily calculated. In calculating percent identity, only exact matches are counted. Methods commonly employed to determine identity or similarity between sequences include, e.g., those disclosed in Carillo et al. (1988), SIAM J. Applied Math. 48, 1073. Preferred methods to determine identity are designed to give the largest match between the sequences tested. Exemplary methods to determine identity and similarity are also provided in commercial computer programs. A preferred example of a mathematical algorithm utilized for the comparison of two sequences is the algorithm of Karlin et al. (1990), Proc. Natl. Acad. Sci. USA 87:2264-2268, modified as in Karlin et al. (1993), Proc. Natl. Acad. Sci. USA 90:5873-5877. Such an algorithm is incorporated into the NBLAST and XBLAST programs of Altschul et al. (1990), J. Mol. Biol. 215:403-410. To obtain gapped alignments for comparison purposes, Gapped BLAST can be utilized as described in Altschul et al. (1997), Nucleic Acids Res. 25:3389-3402. Alternatively, PSI-Blast can be used to perform an iterated search, which detects distant relationships between molecules. When utilizing BLAST, Gapped BLAST, and PSI-Blast programs, the default parameters of the respective programs (e.g., XBLAST and NBLAST) can be used. See, e.g., http://www.ncbi.nlm.nih.gov. Additionally, the FASTA method (Atschul et al. (1990), J. Mol. Biol. 215, 403) can be used. Another preferred example of a mathematical algorithm useful for the comparison of sequences is the algorithm of Myers et al. (1988), CABIOS 4:11-17. Such an algorithm is incorporated into the ALIGN program (version 2.0), which is part of the GCG sequence alignment software package (Devereux et al. (1984), Nucleic Acids Res. 12(1), 387).

The term “substantially similar” as used herein in reference to a polynucleotide or polypeptide sequence includes the identical sequence as well as deletions, substitutions or additions thereto that result in a modified sequence that maintains the desired biological active portion and possesses conserved motifs thereof.

The term “subject” as used herein refers to an animal or human who is the object of treatment, observation or experiment. Preferably, the subject is a mammal, more preferably a human.

The term “substantially pure form” as used herein in reference to a receptor or ligand component means a polypeptide useful as a biological reagent that is essentially free of contaminating matter that would interfere with ligand-receptor binding. Substantially pure forms of polypeptides may be produced by, e.g., isolation, purification, peptide synthesis, or recombinant expression. In exemplary embodiments, the ligand component is produced by isolation or recombinant expression and the receptor component is GPCR142 expressed on the surface of a whole cell. In other embodiments, both the receptor and the ligand component are in substantially pure form so that either of the resulting complexes can be used in applications such as x-ray crystallography to yield a co-crystal structure of such complexes that may be employed in conformational studies or computational modeling to aid in the design of drugs useful in treating disorders mediated by modulation (e.g., agonism or antagonism) of a relaxin3/GPCR142 or an INSL5/GPCR142 interaction.

The term “tag” as used herein refers to an amino acid sequence or a nucleotide sequence encoding an amino acid sequence that facilitates isolation, purification or detection of a protein containing a tag. A variety of such tags are known to those skilled in the art and are suitable for use in the present invention. Suitable tags include, e.g., HA peptide, polyhistidine peptides, biotin/avidin, FLAG, and a variety of antibody epitope binding sites.

The term “therapeutically effective amount” as used herein means that amount of active compound or pharmaceutical agent that elicits the biological or medicinal response in a tissue system, animal or human that is being sought by a researcher, veterinarian, medical doctor or other clinician, such as alleviation of the symptoms of the disease or disorder being treated. Methods are known in the art for routinely determining therapeutically effective doses for pharmaceutical compositions.

The term “vector” refers to a nucleic acid molecule capable of transporting another nucleic acid to which it has been linked. One type of vector is a “plasmid,” which refers to a circular double-stranded DNA loop into which additional DNA segments can be inserted. Another type of vector is a viral vector wherein additional DNA segments can be inserted. Certain vectors are capable of autonomous replication in a host cell into which they are introduced (e. g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors—expression vectors—are capable of directing the expression of genes to which they are operably linked. Vectors of utility in recombinant DNA techniques may be in the form of plasmids. Alternatively, other forms of vectors, such as viral vectors (e.g., replication defective retroviruses, adenoviruses and adeno-associated viruses), which serve equivalent functions, may be used.

Ligand/Receptor Complexes

In preferred embodiments of the ligand-and-receptor complexes of the invention, the ligand component is either a relaxin3 that is originated from a human, a mouse, or a rat, or the ligand component is an INSL5 that is originated from a human. Preferably, the ligand component is either a relaxin3 or an INSL5 that has been recombinantly expressed.

In other preferred embodiments of the ligand-and-receptor complexes of the invention, the ligand component is labeled with a detectable agent, such as a radioisotope or a fluorescent molecule. The labeling technique is selected based on the type of labeling agent employed, and is within the purview of those ordinarily skilled in the art. For instance, labeling can be accomplished by replacing one of the atoms of a ligand molecule with a corresponding radioactive isotope. A hydrogen atom could be replaced with tritium, ³H; a carbon atom can be replaced with carbon-14, ¹⁴C; or a strontium atom can be replaced with strontium-38, ³⁸Sr. In another exemplary labeling process, rather than replacing any atoms of the ligand with a radioactive isotope, an isotope can be added to the ligand molecule. Such radioactive isotopes include, for example, iodine-125, ¹²⁵I, and iron-59, ⁵⁹Fe. In yet another exemplary labeling process, labeling can be carried out by using an appropriate radiolabeled precursor, such as methionine-35 (³⁵S) or phosphate-33 (³³P, for protein phosphorylation), during the synthesis of the peptide either in vivo or in vitro. Preferably, the ligand component of this invention is labeled with iodine-125, ¹²⁵I.

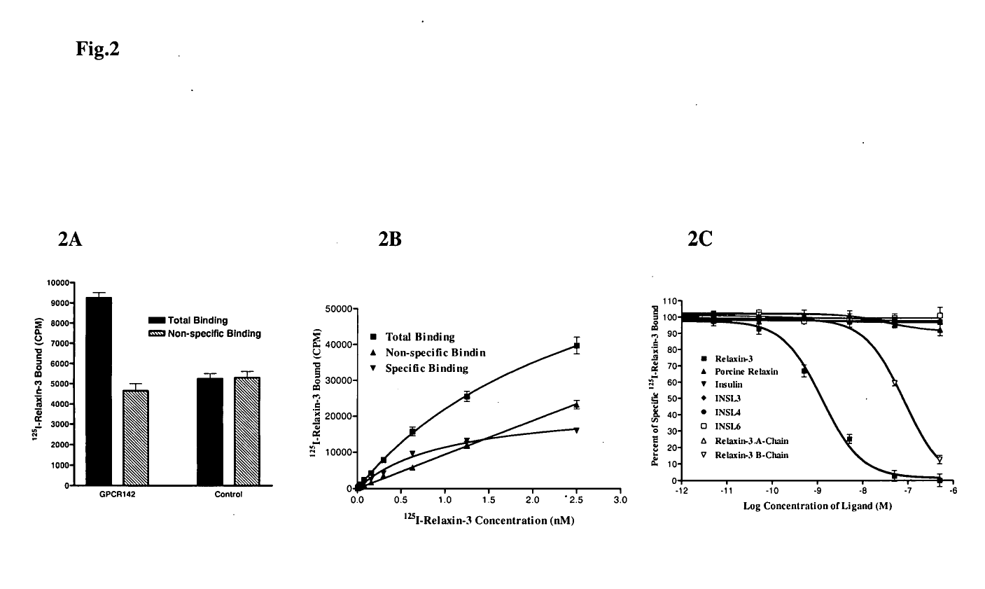

The receptor of the complex may be human GPCR142. To explore other species, mouse and rat genome database information was searched using the human GPCR142 sequence as the query, and the mouse and rat GPCR142 genes identified. The mouse GPCR142 gene was cloned and the open reading frame confirmed by DNA sequencing. The complete coding region of the mouse GPCR142 was deposited in GenBank (GenBank Accession No: AY633765). Overall, the mouse GPCR142 shares 74% sequence identity to that of human. Human GPCR142 has 374 amino acids, whereas the mouse equivalent is longer with 414 amino acids. At the C-termini, the mouse GPCR142 has a different and much longer C-terminal tail than human GPCR142. Searching the rat genomic database, the existence of a rat gene corresponding to GPCR142 was confirmed. However, the rat gene does not have an open reading frame to encode a functional GPCR, and therefore rat GPCR142 appears to be a pseudo-gene. The GPCR142 genes from monkey, cow, and pig were also cloned. DNA sequencing results indicated that GPCR142 genes from monkey, cow, and pig all have opening reading frames and encode putative receptors with high homology to the human GPCR142. The homologies between GPCR142 from different species are shown in FIG. 2. The complete coding regions for GPCR142 from monkey, cow, and pig were deposited in GenBank (Accession Nos. AY633766, AY633767, and AY633768, respectively).

Competition binding experiments were performed to compare the ligand binding properties for recombinant monkey, mouse, bovine, and porcine GPCR142 versus that of the human GPCR142. The results showed that GPCR142 from all species tested binds human relaxin3 with high affinity with IC₅₀ values of 0.87±0.64 nM for bovine, 1.34±0.32 nM for monkey, 1.45±0.22 nM for human, 1.59±0.45 nM for porcine, and 5.45±1 nM for mouse. No specific binding was observed in mock-transfected COS-7 cells. The GPCR142 receptors from different species were also compared in GTPγS binding and Ca²⁺ mobilization assays. In GTPγS binding assays, human relaxin3 potently stimulated all GPCR142 receptors with EC₅₀ values of 0.45±0.07 nM for bovine, 0.96±0.15 nM for porcine, 0.98±0.23 nM for human, 1.1±0.2 nM for monkey, and 3.5±0.76 nM mouse, respectively. Previously, it was shown that relaxin3 stimulates Ca²⁺ signaling in cells expressing human GPCR142 and Gα₁₆ (Liu et al., 2003b). Monkey, bovine, and porcine GPCR142 when co-expressed with Gα₁₆ behaved similarly to that of human GPCR142 with the EC₅₀ values ranging from 50 to 100 nM. However, relaxin3 did not stimulate detectable Ca²⁺ response in cells expressing mouse GPCR142 and Gα₁₆.

Accordingly, the ligand and receptor complexes of the invention comprise a GPCR142 that is preferably originated from a human, mouse, monkey, cow, or pig. Thus, in a preferred embodiment, the GPCR142 polypeptide is selected from SEQ ID NO: 4, SEQ ID NO:2 , SEQ ID NO:7, SEQ ID NO:8, or SEQ ID NO: 9.

In a certain preferred embodiment, the GPCR142 is expressed on the cell surface of a GPCR142 host cell, preferably a recombinant GPCR142 host cell. In another preferred embodiment, the GPCR142 is associated with isolated cell membranes from a GPCR142 host cell, preferably from a recombinant GPCR142 host cell. In yet another preferred embodiment, the receptor component of the complex is a fragment of the GPCR142 capable of binding to a relaxin3 ligand or an INSL5 ligand.

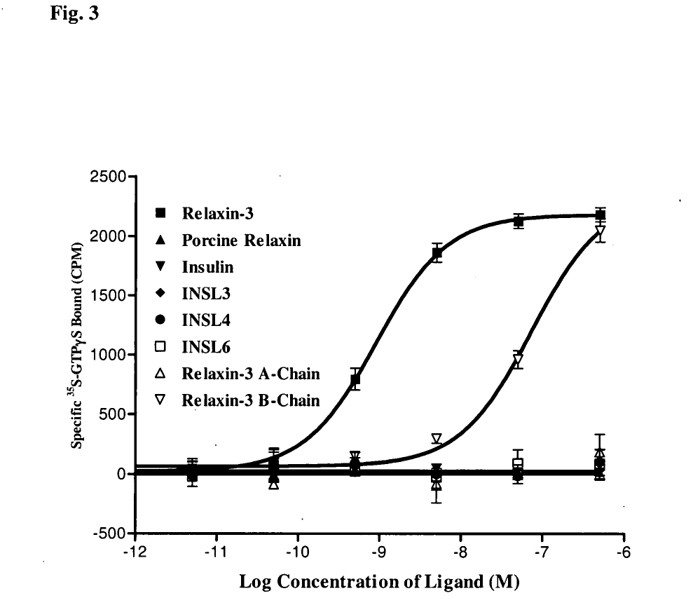

The ligand component of the complex can be either a full-length relaxin3 or an active fragment thereof that is still capable of binding to a GPCR142. As apparent from the results shown in FIGS. 2C and 3, the B-chain is capable of binding and activating GPCR142. The ligand component of the complex also can be either a full-length INSL5 or an active fragment thereof that is still capable of binding to a GPCR142.

Any suitable methods for constituting a ligand component and receptor complex known to the skilled artisan may be used to form such a complex. In general, the method comprises mixing a sample comprising the ligand component with a sample comprising the receptor.

The sample comprising the ligand component can be tissue or cell extract containing the ligand component, or purified ligand component. This sample can be prepared from a natural source of the ligand component, e.g., an endogenous host cell or tissue for the ligand component of warm-blooded animals inclusive of human. Preferably, the sample comprising the ligand component is prepared from a recombinant host cell that expresses increased amount of the ligand component. A recombinant host cell for the ligand component may be constructed by introducing into the cell a DNA molecule capable of expressing the functional ligand component.

In the production from the tissues or cells of human or other warm-blooded animals, the ligand component polypeptide can be purified and isolated by a process comprising homogenizing the tissue or cells of human or other warm-blooded animal, extracting the homogenate with an acid or another suitable extracting agent, and isolating the ligand component polypeptide from the extract, e.g., via a combination of chromatographic procedures, such as reversed-phase chromatography, ion-exchange chromatography, and affinity chromatography.

The ligand component in the present invention can also be produced by known procedures for peptide synthesis. The methods for peptide synthesis may be selected from suitable solid-phase synthesis and liquid-phase synthesis techniques. For example, the desired peptide can be produced by condensing a partial peptide or amino acid capable of constituting the protein with the residual part thereof and, when the product has a protective group, removing the protective group. Methods for condensation and deprotection during peptide synthesis are described in literature, for example, in: Bodanszky and Ondetti, “Peptide Synthesis,” Interscience Publishers, New York, 1966; Schroeder and Luebke, “The Peptide,” Academic Press, New York, 1965; Izumiya et al., “Fundamentals and Experiments in Peptide Synthesis,” Maruzen, 1975; Yajima and Sakakibara, “Biochemical Experiment Series 1, Protein Chemistry IV,” 205, 1977; and Yajima (ed.), “Development of Drugs-Continued, 14, Peptide Synthesis,” Hirokawa Shoten.

After the peptide synthesis reaction, the protein product can be purified and isolated by a suitable combination of conventional purification techniques, such as solvent extraction, column chromatography, liquid chromatography, and recrystallization. Where the protein isolated is in a free form, it can be converted to a suitable salt by a known method. Conversely, where the isolated product is a salt, it can be converted to the free peptide by a suitable method selected from those known in the art.

The amide of a polypeptide can be obtained by using a resin for peptide synthesis that is suited for amidation. Exemplary resins include chloromethyl resin, hydroxymethyl resin, benzhydrylamine resin, aminomethyl resin, 4-benzyloxybenzyl alcohol resin, 4-methylbenz-hydrylamine resin, PAM resin, 4-hydroxymethylmethylphenyl-acetamidomethyl resin, polyacrylamide resin, 4-(2′,4′-dimethoxyphenyl-hydroxymethyl)phenoxy resin, and 4-(2′,4′-dimethoxyphenyl-Fmoc aminoethyl)phenoxy resin. Using such a resin, amino acids whose a-amino groups and functional side-chain groups (R groups) have been suitably protected are condensed on the resin according to the sequence of the objective peptide by various condensation techniques that are known to those skilled in the art. At the end of the series of reactions, the peptide or the protected peptide is separated from the resin and the protective groups are removed to obtain the objective polypeptide.

The sample comprising the GPCR142 receptor can comprise intact host cells with the receptor expressed on the cell-surface, isolated cell membranes from host cells of the receptor, or purified fragment of the receptor that is capable of binding to the ligand. Although an endogenous host cell for GPCR142 receptor can be used, a recombinant host cell expressing an increased amount of GPCR142 on the cell surface is preferred.

It is known that a GPCR binds to its endogenous ligand sometimes with its extracellular domain. Such a binding domain can be identified by various methods known to those skilled in the art, such as sequence analyses, protein-protein interaction analyses, protein structural analyses, or a combination of these methods. For example, the ligand binding domain in metabotropic glutamate receptors has been identified as a Venus flytrap module (VFTM) in its extracellular domain (O'Hara et al., 1993, Neuron, 11(1): 41-52; David et al., 1999, J. Biol. Chem., 274: 33488-33495). In a preferred embodiment, the ligand-component binding domain of GPCR142 can be first identified using the above methods, and such a ligand-component binding domain can be recombinantly expressed, purified and used in forming a complex of the invention.

GPCR142 of the desired species may be cloned using techniques such as those described in the examples below. Any of a variety of procedures known in the art can be used to isolate the nucleic acid molecule of the invention. For example, using cDNA or genomic DNA libraries, or total mRNA from the suitable cells identified above as a template and appropriate oligonucleotide as primers, a nucleic acid molecule of the invention can be amplified according to standard PCR amplification techniques. The nucleic acid so amplified from PCR can be cloned into an appropriate vector and characterized by DNA sequence analysis. Primers can be prepared by standard synthetic techniques, e.g., using an automated DNA synthesizer.

Another method to isolate a nucleic acid molecule of the invention is to probe a genomic or cDNA library, or total mRNA with one or more natural or artificially designed probes using procedures available to those skilled in the art. See, e.g., “Current Protocols in Molecular Biology”, Ausubel et al.(eds.), Greene Publishing Association and John Wiley Interscience, New York, 1992. Preferred probes will have from 30 to 50 bases. Such probes can be labeled with an analytically detectable reagent to facilitate identification of the probe. Useful reagents include, e.g., radioisotopes, fluorescent dyes, or enzymes capable of catalyzing the formation of a detectable product. The probes enable the ordinarily skilled artisan to isolate complementary copies of genomic DNA, cDNA or RNA polynucleotides encoding GPCR142, such as mouse as well as human, rat, monkey, cow, and pig.

Another method to prepare nucleic acid molecules is by standard synthetic techniques, e.g., using an automated DNA synthesizer. Construction of genomic DNA libraries, preparation of cDNA libraries, or isolation of total mRNA from the identified source cell can be performed by standard techniques known in the art. These techniques can be found, for example, in Maniatis et al., “Molecular Cloning: A Laboratory Manual,” 2^{nd} ed. (Cold Spring Harbor Laboratory, Cold Spring Harbor, N.Y., 1989).

In another aspect, the present invention provides vectors, preferably expression vectors, containing a nucleic acid that is capable of expressing mouse GPCR142. The recombinant expression vectors of the invention comprise a nucleic acid of the invention in a form suitable for expression of the nucleic acid in a host cell. Thus, the recombinant expression vectors include one or more regulatory sequences, selected on the basis of the host cells to be used for expression, operably linked to the nucleic acid sequence to be expressed. When used in reference to a recombinant expression vector, “operably linked” is intended to mean that the nucleotide sequence of interest is linked to the regulatory sequence(s) in a manner allowing for expression of the nucleotide sequence (e.g., in an in vitro transcription/translation system or in a host cell when the vector is introduced into the host cell). It will be appreciated by those of ordinary skill in the art that the design of the expression vector can depend on such factors as the choice of the host cell to be transformed and the level of expression of protein desired. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., Escherichia coli (E. coli)) or eukaryotic cells (e.g., insect cells (using baculovirus expression vectors), yeast cells or mammalian cells). Suitable host cells may be routinely determined. Alternatively, the recombinant expression vector can be transcribed and translated in vitro, for example using T7 promoter regulatory sequences and T7 polymerase.

Expression of proteins in prokaryotes may be carried out in E. coli with vectors containing constitutive or inducible promoters directing the expression of either fusion or non-fusion proteins. Fusion vectors add a number of amino acids to a protein, usually to the amino terminus of the recombinant protein. Such fusion vectors typically are used for one or more of the following purposes: to increase expression of recombinant protein; to increase the solubility of the recombinant protein; to aid in the purification of the recombinant protein by acting as a ligand in affinity purification; and to facilitate detection of the recombinant protein by serving as a marker. Often in fusion expression vectors, a proteolytic cleavage site is introduced at the junction of the fusion moiety and the recombinant protein to enable separation of the recombinant protein from the fusion moiety subsequent to purification of the fusion protein. Such enzymes and their cognate recognition sequences include Factor Xa, thrombin and enterokinase. Typical fusion expression vectors include PGEX (Pharmacia Biotech Inc; Smith et al., (1988), Gene 67:31-40), pMAL (New England Biolabs, Beverly, Mass.), pRIT5 (Pharmacia, Piscataway, N.J.), or pQE (Qiagen), which fuse glutathione S-transferase (GST), maltose binding protein, protein A, or poly-His, respectively, to the target recombinant protein.

Examples of suitable inducible non-fusion E. coli expression vectors include pTrc (Amann et al., (1988), Gene 69:301-315) and pETIId (Studier et al., “Gene Expression Technology: Methods in Enzymology 185,” Academic Press, San Diego, Calif. (1990) 60-89). One strategy to maximize recombinant protein expression in E. coli is to express the protein in a host bacterium with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention can be carried out by standard DNA synthesis techniques.

In another preferred embodiment, the expression vector is a yeast expression vector. Examples of vectors for expression in yeast S. cerivisae include pYepSecl (Baldari et al. (1987), EMBO J 6:229-234), pMFa (KurJan et al. (1982), Cell 30:933-943), pJRY88 (Schultz et al. (1987), Gene 54:113-123), pYES2 (Invitrogen Corporation, San Diego, Calif.), and pPicZ or Pichia (Invitrogen Corp, San Diego, Calif.).

Alternatively, the expression vector is a baculovirus expression vector. Baculovirus vectors available for expression of proteins in cultured insect cells include, e.g., the pAc series (Smith et al. (1983), Mol. Cell. Biol. 3:2156-2165) and the pVL series (Lucklow et al. (1989), Virology 170:31-39). Commercially available insect cell expression vectors useful for recombinant expression include pBlueBacII (Invitrogen).

In yet another preferred embodiment, the expression vector is a mammalian expression vector. When used in mammalian cells, the expression vector's control functions are often provided by viral regulatory elements. For example, commonly used promoters are derived from polyoma, Adenovirus 2, cytomegalovirus and Simian Virus 40. Examples of mammalian expression vectors include, e.g., pCDM8 (Seed (1987), Nature 329:840) and pMT2PC (Kaufinan et al., (1987), EMBO J 6:187-195). Commercially available mammalian expression vectors which can be suitable for recombinant protease COX-3 expression include, for example, pMAMneo (Clontech), pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and 1ZD35 (ATCC 37565).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Examples of suitable tissue-specific promoters include the albumin promoter (liver-specific; Pinkert et al. (1987), Genes Dev. 1:268-277), lymphoid-specific promoters (Calame et al. (1988), Adv. Immunol. 43:235-275), in particular promoters of T cell receptors (Winoto et al. (1989), EMBO J. 8:729-733) and immunoglobulins (BaneiJi et al. (1983), Cell 33:729-740; Queen et al., (1983), Cell 33:741-748), neuron-specific promoters (e.g., the neurofilament promoter; Byme et al. (1989), Proc. Natl. Acad. Sci. USA 86:5473-5477), pancreas-specific promoters (Edlund et al. (1985), Science 230:912-916), and mammary gland-specific promoters (e.g., milk whey promoter; U.S. Pat. No. 4,873,316 and European Patent Publication No. 264,166). Developmentally regulated promoters also include, for example, the marine hox promoters (Kessel et al. (1990), Science 249:374-379) and the beta-fetoprotein promoter (Campes et al. (1989), Genes Dev. 3:537-546).

The invention further provides a recombinant vector comprising a DNA molecule of the invention cloned into the vector in an antisense orientation. That is, the DNA molecule is operably linked to a regulatory sequence in a manner that allows for expression (by transcription of the DNA molecule) of an RNA molecule that is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell-type specific expression of antisense RNA. The antisense expression vector can be in the form of a recombinant plasmid, phagemid or attenuated virus in which antisense nucleic acids are produced under the control of a high-efficiency regulatory region, the activity of which can be determined by the cell type into which the vector is introduced. For a discussion of the regulation of gene expression using antisense genes see Weintraub et al. (“Reviews—Trends in Genetics,” Vol. 1(1) 1986).

The invention also provides in a preferred embodiment a recombinant vector system that directs the synthesis of small interfering RNAs (siRNAs) in mammalian cells. Many organisms possess mechanisms to silence any gene expression when double-stranded RNA (dsRNA) corresponding to the gene is present in the cell through a process known as RNA interference. The technique of using dsRNA to reduce the activity of a specific gene was first developed using the worm C. elegans and has been termed RNA interference or RNAi (Fire et al. (1998), Nature 391: 806-811). RNAi has since been found to be useful in many organisms, and recently has been extended to mammalian cells in culture (see review by Moss (2001), Curr Biol 11: R772-5). An important advance was made when RNAi was shown to involve the generation of small RNAs of 21-25 nucleotides (Hammond et al. (2000), Nature 404: 293-6; Zamore et al., (2000) Cell 101: 25-33). These small interfering RNAs, or siRNAs, may initially be derived from a larger dsRNA that begins the process, and are complementary to the target RNA that is eventually degraded. The siRNAs are themselves double-stranded with short overhangs at each end; they act as guide RNAs, directing a single cleavage of the target in the region of complementarity (Elbashir et al. (2001), Genes Dev. 15: 188-200; Zamore et al. (2000), Cell 101: 25-33).

An siRNA comprising nucleotides that are complementary to mouse GPCR142 may be produced in vitro, for example, using a method described in WIPO Publication No. WO 01/75164, or can be produced in vivo from a mammalian cell using a stable expression system. An exemplary vector system that directs the synthesis of siRNAs in mammalian cells is the pSUPER (Brummelkamp et al. (2002), Science 296: 550-3).

Exemplary vectors of the present invention also include specifically designed vectors that allow the shuttling of DNA between hosts such as bacteria-yeast or bacteria-animal cells or bacteria-fungal cells or bacteria-invertebrate cells. Numerous cloning vectors are known to those skilled in the art and the selection of an appropriate cloning vector is within the purview of the artisan. For other suitable expression systems for both prokaryotic and eukaryotic cells see, e.g., chapters 16 and 17 of Maniatis et al., supra.

The present invention also provides recombinant host cells into which a recombinant vector of the invention has been introduced. Cell lines derived from mammalian species which can be suitable for transfection and which are commercially available include, e.g., CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), Drosophila or murine L-cells, and HEK-293 (ATCC CRL1573), and monkey kidney cells.

Vector DNA can be introduced into prokaryotic or eukaryotic cells via conventional transformation or transfection techniques. As used herein, the term “transformation” or “transfection” refers to a process by which cells take up foreign DNA and may or may not integrate that foreign DNA into their chromosome. Transfection can be accomplished, for example, by various techniques including calcium phosphate or calcium chloride co-precipitation, DEAE-dextran-mediated transfection, lipofection, electroporation, or protoplast fusion. Suitable methods for transforming or transfecting host cells can be found, e.g., in Maniatis et al. (supra).

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) may be introduced into the host cells along with the gene of interest. Preferred selectable markers include those that confer resistance to drugs, such as G418, hygromycin and methotrexate. Cells stably transfected with the introduced nucleic acid can be identified by drug selection (e.g., cells that have incorporated the selectable marker gene will survive, while the other cells die).

In another embodiment, the expression characteristics of an endogenous nucleic acid within a cell, cell line or microorganism can be modified by inserting a DNA regulatory element heterologous to the endogenous gene of interest into the genome of a cell, a stable cell line or a cloned microorganism such that the inserted regulatory element is operatively linked with the endogenous gene and controls, modulates or activates the endogenous gene. A heterologous regulatory element can be inserted into a stable cell line or cloned microorganism such that it is operatively linked with and activates expression of endogenous genes, using techniques such as targeted homologous recombination, e.g., as described in U.S. Pat. No. 5,272,071 and WIPO Publication No. WO 91/06667.

A preferred embodiment of the invention provides a substantially purified polypeptide consisting essentially of the amino acid sequence of SEQ ID NO: 7, 8, or 9. The invention also pertains to methods of expressing or isolating the inventive polypeptide.

In one embodiment, the polypeptide can be isolated from cells or tissue sources that express it naturally by an appropriate purification scheme using standard protein purification techniques. In another embodiment, polypeptides of the invention are produced by recombinant DNA techniques. Alternatively, a polypeptide of the invention can be synthesized in an in vitro translation and/or transcription system. Further alternatively, a polypeptide of the invention can be synthesized chemically using standard peptide synthesis techniques.

Polypeptides of the invention can be recombinantly expressed by cloning DNA molecules of the invention into an expression vector described above, introducing such a vector into prokaryotic or eukaryotic host cells as described herein, and growing the host cell under conditions suitable for production of recombinant protein. The expression vector-containing cells are clonally propagated and individually analyzed to determine whether they produce the polypeptide of the invention. Identification of the animal GPCR142-expressing host cell clones can be done by several means, including immunological reactivity with anti-animal GPCR142 antibodies, and the presence of host cell-associated GPCR142 activity, such as relaxin3 binding or INSL5 binding. The selection of the appropriate growth conditions and recovery methods are within the skill of the art. Techniques for recombinantly expressing a polypeptide are described in, e.g., Maniatis et al., supra.

Polypeptides of the invention can also be produced using an in vitro translation and/or transcription system known in the art. For example, synthetic monkey, rat, bovine, or porcine GPCR142 mRNA or mRNA isolated from cells can be efficiently translated in various cell-free systems, including wheat germ extracts and reticulocyte extracts. Alternatively, the coding sequence of the monkey, rat, bovine, or porcine GPCR142 cDNA can be cloned under the control of a T7 promoter. Then, using this construct as the template, the protein can be produced in an in vitro transcription and translation system, for example using a TNT T7 coupled Reticulocyte Lysate System such as that commercially available from Promega (Madison, Wis.).

Polypeptides of the invention can also be produced by chemical synthesis, using methods as described supra.

The mouse GPCR142 protein can be purified by methods known to those skilled in the art. For example, it can be purified from cell lysates and extracts from natural or recombinant host cells, by various combinations or individual application of salt fractionation, ion exchange chromatography, size exclusion chromatography, hydroxylapatite adsorption chromatography and hydrophobic interaction chromatography, lectin chromatography, HPLC, and FPLC, and antibody/ligand affinity chromatography.

Production of A Relaxin3 from A Recombinant Cell

Because relaxin3 is a secreted protein composed of two chains (A-chain and B-chain) of polypeptides linked by disulfide bridges, proteases are involved in the production of mature relaxin3 from its pre-propeptide. Signal sequence peptidase cleaves off the signal sequence from the propeptide, resulting in relaxin3 propeptide. The propeptide is subsequently cleaved into chains A, B, and C of the relaxin3 by pro-hormone convertases. A-chain and B-chain form the final mature relaxin3, and the C-chain is not part of the mature relaxin3. Because pro-hormone convertases are only selectively expressed in certain cell types where hormones are secreted, such as some neuron cells or endocrine cells, the relaxin3 propeptide is often not efficiently processed when it is recombinantly expressed from other cell type, such as a COS7.

Relaxin3 may be produced from a recombinant cell by steps comprising: constructing a DNA molecule capable of encoding a modified relaxin3 propeptide with a protease cleavage site inserted at the peptide junction between chains A and C, and/or chains C and B of the relaxin3 propeptide; constructing a vector capable of expressing the modified relaxin3 propeptide; constructing another vector capable of expressing a protease that can cleave the modified relaxin3 propeptide at the inserted protease cleavage site(s); introducing both the vectors into a host cell; and growing the host cell under conditions suitable for the expression of both the modified relaxin3 propeptide and the protease, so that the protease will efficiently cleave the peptide linkage between chains A and C, or chains C and B on the modified relaxin3 propeptide.

Sequence analyses suggested that furin, a member of the pro-hormone convertases, cleaves the peptide linkage between chains C and B on the native relaxin3 propeptide, and another unidentified protease cleaves the peptide linkage between chains A and C on the native propeptide. A furin cleavage site, with the amino acid sequence of arg-gly-arg-arg (RGRR), may be inserted at the peptide junction of chains A and C. An expression vector for this mutant relaxin3 and another expression vector for furin are co-transfected into a host cell. Under suitable growth conditions, the relaxin3 pro-peptide is almost completely processed into mature peptides, A, B, and C. Instead of furin, other proteases can also be used. Identical protease cleavage sites can be inserted between chains A and C, and chains C and B, on the pro-peptide. Examples of proteases that can be used include, e.g., a pro-hormone convertase, such as furin, PC1, and PC2 (Hosaka et. al., 1991, J. Biol. Chem. 266:12127-12130; Benjannet et. al., 1991, Proc. Natl. Acad. Sci. USA, 88:3564-3568; Thomas et. al., 1991, Proc. Natl. Acad. Sci. USA, 88:5297-5301).

Because of its high homology to relaxin3, it was speculated that INSL5 could be an additional ligand for GPCR135, GPCR142, LGR7, or LGR8. Accordingly, human INSL5 peptides were recombinantly expressed. It was found that INSL5 is an agonist for GPCR142 but not for GPCR135.

The identified interaction between GPCR142 and INSL5 allows for development of transgenic animals, such as knock-out mice, in which a GPCR142 gene and/or INSL5 gene has been introduced or disrupted. The identified interaction between the receptor and ligand also allows for the employment of GPCR142, or a GPCR142/INSL5 complex, in screening methods or assays for identifying compounds for their potential efficacy in treating a disorder related to the INSL5 and GPCR142 complex.

In one preferred embodiment, a tag, such as a HA, poly His, or FLAG, can be added either to the modified relaxin3 propeptide or to the INSL5 to facilitate protein isolation or purification. Techniques known to modify a DNA molecule to cause certain desirable changes in the amino acid sequence encoded by such DNA molecule, such as PCR, may be employed.

Method of Identifying Modulators of The Activity of Either The Relaxin3/GPCR142 or The INSL5/GPCR142 Complex

Another general aspect of the invention relates to a method of identifying modulators that either increase or decrease biological activity of either a relaxin3/GPCR142 complex or a INSL5/GPCR142 complex. Such modulators should be useful as therapeutic agents in treating a subject suffering from a disease or disorder related to either the relaxin3/GPCR142 complex or the INSL5/GPCR142 complex, such as CNS disorders (anxiety, schizophrenia, depression, mood, sleep/wake), metabolic disorders, feeding/drinking disorders, water and nutrient homeostasis, and endocrine disorders (see Goto et al., 2001, J. Comp. Neurol. 438: 86-122).

“Inhibitors” refer to compounds that decrease, prevent, inactivate, desensitize or down-regulate either relaxin3/GPCR142 complex expression or activity or INSL5/GPCR142 complex expression or activity. “Activators” are compounds that increase, activate, facilitate, sensitize or up-regulate relaxin3/GPCR142 complex expression or activity or INSL5/GPCR142 complex expression or activity. “Modulators” include both “inhibitors” and “activators”.

The compound identification methods can be performed using conventional laboratory formats or in assays adapted for high throughput. The term “high throughput” refers to an assay design that allows easy screening of multiple samples simultaneously or single samples rapidly, and can include the capacity for robotic manipulation. Another desired feature of high throughput assays is an assay design that is optimized to reduce reagent usage, or minimize the number of manipulations in order to achieve the analysis desired. Examples of assay formats include 96-well or 384-well plates, levitating droplets, and “lab on a chip” microchannel chips used for liquid-handling experiments. Of course, as miniaturization of plastic molds and liquid-handling devices are advanced, or as improved assay devices are designed, greater numbers of samples will be able to be screened more efficiently using the inventive assay.

Candidate compounds for screening can be selected from numerous chemical classes, preferably from classes of organic compounds. Although candidate compounds can be macromolecules, preferably the candidate compounds are small-molecule organic compounds, i.e., those having a molecular weight of greater than 50 and less than 2500. Candidate compounds have one or more functional chemical groups necessary for structural interactions with polypeptides. Preferred candidate compounds have at least an amine, carbonyl, hydroxyl or carboxyl group, preferably at least two such functional groups, and more preferably at least three such functional groups. The candidate compounds can comprise cyclic carbon or heterocyclic structural moieties and/or aromatic or polyaromatic structural moieties substituted with one or more of the above-exemplified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound is preferably a DNA or RNA molecule, although modified nucleic acids having non-natural bonds or subunits are also contemplated.

Candidate compounds may be obtained from a variety of sources, including libraries of synthetic or natural compounds. For example, numerous means are available for random and directed synthesis of a variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including: biological libraries; spatially addressable parallel solid-phase or solution-phase libraries; synthetic library methods requiring deconvolution; the “one-bead one-compound” library method; and synthetic library methods using affinity chromatography selection (see, e.g., Lam (1997), Anti-Cancer Drug Des. 12:145). Alternatively, libraries of natural compounds in the form of bacterial, fungal, plant and animal extracts are available or may be routinely produced. Additionally, natural and synthetically produced libraries and compounds can be routinely modified through conventional chemical, physical, and biochemical means.

Furthermore, known pharmacological agents can be subjected to directed or random chemical modifications, such as acylation, alkylation, esterification, and amidification to produce structural analogs of the agents. Candidate compounds can be selected randomly or can be based on existing compounds that bind to and/or modulate the function or activity of a GPCR. Therefore, a source of candidate agents is known or screened libraries of molecules including activators or inhibitors of GPCRs with similar structures to GPCR142. The structures of such compounds may be changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions.

A variety of other reagents also can be included in the assay mixture. These include reagents such as salts, buffers, neutral proteins (e.g., albumin), and detergents that can be used to facilitate optimal protein-protein and/or protein-nucleic acid binding. Such a reagent can also reduce non-specific or background interactions of the reaction components. Other reagents that improve the efficiency of the assay, such as nuclease inhibitors, antimicrobial agents, and the like, can also be used.

Examples of methods for the synthesis of molecular libraries can be found in the art, for example in Zuckermann et al. (1994), J. Med. Chem. 37:2678. Libraries of compounds can be presented in solution (e.g., Houghten (1992), Biotechniques 13:412-421), or on beads (Lam (1991), Nature 354:82-84), chips (Fodor (1993), Nature 364:555-556), bacteria (U.S. Pat. No. 5,223,409), spores (U.S. Pat. No. 5,571,698), plasmids (Cull et al. (1992), Proc. Natl. Acad. Sci. USA 89:1865-1869) or phage (see e.g., Scott and Smith (1990), Science 249:386-390).

Thus, in one general aspect the invention relates to a method of identifying a compound that increases or decreases a biological activity of a relaxin3/GPCR142 complex, comprising the steps of:
(a) contacting a solution comprising a buffer and a candidate or test compound with an assay reagent comprising the relaxin3 and GPCR142 complex;
(b) measuring the biological activity of the relaxin3 and GPCR142 complex; and
(c) comparing the result of step(b) with that of a control wherein the relaxin3 and GPCR142 complex was contacted with only the buffer.

In a preferred embodiment, the relaxin3 and GPCR142 complex in the method is associated with a cell expressing the GPCR142 on the cell surface.

Another general aspect the invention relates to a method of identifying a compound that increases or decreases a biological activity of an INSL5/GPCR142 complex, comprising the steps of:
(a) contacting a solution comprising a buffer and a candidate or test compound with an assay reagent comprising the INSL5 and GPCR142 complex;
(b) measuring the biological activity of the INSL5 and GPCR142 complex; and
(c) comparing the result of step(b) with that of a control wherein the INSL5 and GPCR142 complex was contacted with only the buffer.

In a preferred embodiment, the INSL5 and GPCR142 complex in the method is associated with a cell expressing the GPCR142 on the cell surface.

The term “cell” refers to at least one cell or a plurality of cells appropriate for the sensitivity of the detection method. Cells suitable for the present invention may be bacterial, but are preferably eukaryotic, such as yeast, insect, or mammalian. The cell can be a natural host cell for an endogenous GPCR142, preferably a recombinant host cell for a GPCR142, which expresses a high amount of a mammalian GPCR142 on the cell surface.

Either the relaxin3/GPCR142 complex or the INSL5/GPCR142 complex can be formed by adding either a relaxin3 or an INSL5, respectively, to the GPCR142 host cell in the form of a purified protein, or in the form of a cell or tissue extract containing either the relaxin3 or the INSL5. Either the relaxin3 or the INSL5 can be the full-length mature polypeptide, or a fragment that is still capable of binding to a GPCR142.

In preferred embodiments, the biological activity of the inventive ligand component/GPCR142 complexes can be measured by a second messenger response of the cell. For example, the biological activity of the complex can be measured by the signal transduction event triggered by activated GPCR142. This signal transduction event can be measured indirectly by means of measuring one or more changes in cellular physiology, such as cell morphology, migration, or chemotaxis, using one or more suitable methods known in the art. It can also be measured directly by measuring phosphorylation of proteins involved in the signal transduction pathway, for example, the phosphorylation of a GTP-binding protein (G protein). Methods are known in the art for measuring protein phosphorylation, for example, by using an ATP or GTP molecule that has been radiolabeled on the γ-phosphate.

The biological activity of the inventive ligand component/GPCR142 complexes can also be measured by the intracellular concentration of a second messenger molecule using any of a number of suitable techniques known in the art. For example, the pH change can be measured using a pH-sensitive dye, such as Acridine Orange. The calcium concentration can be measured via optical imaging of fluorescent indicators sensitive to Ca²⁺, such as fluo-3 (pentapotassium salt, cell-impermeant form; Molecular Probes) or fluo-3(AM) (an acetoxymethyl ester form of fluo-3, Teflabs) (see for example, Liu et al., 2001, J. Pharmacol. Exp. Ther. 299: 121-30) using a fluorometric imaging plate reader (FLIPR) or a confocal microscope. The cAMP concentration can be detected using a commercially available ELISA kit (FLASHPLATE cyclic AMP assay system (¹²⁵I), Cat. No: SMP001A, NEN; see also Shimomura et al., 2002, J. Biol. Chem. 277: 35826-32), or via a reporter system wherein the expression of a reporter gene, such as beta-galactosidase, is under the control of a cAMP responsive element (cre) (Montminy et al., 1990, Trends Neurosci., 13(5): 184-8).

The test compound can be further characterized by comparing its effect on two cells, the first cell containing a functional GPCR142 and the second one identical to the first, but lacking a functional GPCR142. This technique is also useful in establishing the background noise of these assays. One of ordinary skill in the art will appreciate that this control mechanism also allows ready selection of cellular changes that are responsive to modulation of functional GPCR142. Therefore, in preferred embodiments, the screening method comprises the steps of: (a) contacting a first cell having a GPCR142 expressed on the cell surface with a ligand component and with a test compound; (b) determining a second messenger response in the first cell to the test compound, and comparing it with that of a control wherein the first cell is only contacted with the ligand component, but not the test compound; (c) contacting a second cell with the ligand component and with a test compound; wherein the second cell is otherwise identical to the first cell except that it does not express a GPCR142 on the cell surface; (d) determining a second messenger response of the second cell to the test compound, and comparing the second messenger response with that of a control wherein the second cell is only contacted with the ligand component, but not the test compound; and (e) comparing the comparison result of (b) with that of (d).

There are a number of ways to obtain two cells that are otherwise identical except that one expresses a GPCR142 on its cell surface and the other does not. In one embodiment, the first cell is a recombinant host cell for GPCR142 that constitutively expresses GPCR142 on its cell surface, and the second cell is the parent cell from which the GPCR142 recombinant cell is constructed. In another embodiment, a recombinant host cell for GPCR142 is constructed such that its expression on the cell surface is under the control of an inducible promoter. The first cell is the recombinant cell grown under inducible conditions that allows the expression of GPCR142 on its cell surface, and the second cell is the recombinant cell grown under non-inducible conditions that do not allow the expression of GPCR142. In yet another embodiment, the first cell is a native host cell for GPCR142 that expresses the polypeptide on its cell surface, and the second cell is a mutant cell derived from the native host, wherein the GPCR142 gene has been inactivated through mutagenesis. Standard molecular biology methods can be used to construct a recombinant host cell for GPCR142, or to inactivate a GPCR142 gene.

In other preferred embodiments, the present invention provides a method of identifying a compound that increases or decreases the activity of a ligand component/GPCE142 complex, comprising the steps of: (a) contacting an isolated membrane preparation comprising a GPCR142 with a ligand component with a test compound, and with a GTP molecule that has been labeled on the γ-phosphate; and (b) determining the amount of labeling bound to the membrane preparation; and (c) comparing the amount of labeling in (b) with that of a control wherein the membrane preparation is only contacted with the ligand component and the labeled GTP but not the test compound.

The membrane preparation can be isolated from a native host cell that expresses GPCR142 on its cell surface, or preferably, from a recombinant host cell that expresses increased amount of GPCR142 on its cell surface. It can also be isolated from tissues comprising GPCR142 host cells.

A variety of labels can be used to label the GTP molecule on the γ-phosphate, such as a fluorescent molecule or a radioactive isotope such as ³⁵S, ³²P, and the like.

In yet other embodiments, the present invention provides a method of identifying a compound that binds to a GPCR142, comprising the steps of: a) contacting a GPCR142 with a test compound and with a labeled ligand component; b) measuring the amount of the labeled ligand component that binds to the GPCR142; and c) comparing the measured amount of (b) with that of a control, wherein the GPCR142 is only contacted with a labeled ligand component, but not the test compound.

In one preferred embodiment, a GPCR142 host cell (recombinant or native) that expresses the GPCR142 on the cell surface can be used for the binding assay. In another preferred embodiment, isolated membrane preparations comprising the GPCR142 can be used for the binding assay. In yet another preferred embodiment, a substantially purified extracellular fragment of GPCR142 that is capable of binding to a relaxin3 can be used for the binding assay.

The amount of the labeled ligand component or fragment thereof that binds to the GPCR142 can be measured by first separating the unbound labeled ligand component or fragment from the GPCR142, and then measuring the amount of labeling that is associated with the GPCR142.

Separation of the GPCR142 protein from unbound labeled ligand components or fragments thereof can be accomplished in a variety of ways. Conveniently, the GPCR142 may be immobilized on a solid substrate, from which the ligand component can be easily separated. The solid substrate can be made of a variety of materials and in a variety of shapes, e.g., microtiter plate, microbead, dipstick, and resin particle. The substrate preferably is chosen to maximize signal-to-noise ratios, primarily to minimize background binding, as well as for ease of separation and cost.

Separation can be effected by, for example, removing a bead or dipstick from a reservoir, emptying or diluting a reservoir such as a microtiter plate well, or rinsing a bead, particle, chromatographic column or filter with a wash solution or solvent. The separation step preferably includes multiple rinses or washes. For example, when the solid substrate is a microtiter plate, the wells can be washed several times with a washing solution, e.g., that includes those components of the incubation mixture that do not participate in specific bindings, such as salts, buffer, detergent, non-specific protein, etc. Where the solid substrate is a magnetic bead, the beads can be washed one or more times with a washing solution and isolated using a magnet.

GPCR142 can be immobilized on a solid substrate using a number of methods. In one embodiment, a fusion protein can be provided which adds a domain that allows the GPCR142 proteins to be bound to a matrix. For example, glutathione-S-transferase fusion proteins or glutathione-S-transferase fusion proteins can be adsorbed onto glutathione sepharose beads (Sigma Chemical, St. Louis, Mo.) or glutathione derivatized microtiter plates, which are then combined with the test compound and the labeled ligand component, and the mixture incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding of the labeled ligand component to GPCR142 can be determined using standard techniques.

Other techniques for immobilizing proteins on matrices can also be used in the screening assays of the invention. For example, the GPCR142 can be immobilized utilizing conjugation of biotin and streptavidin. Biotinylated polypeptide can be prepared from biotin-NHS (N-hydroxy-succinimide) using techniques known in the art (e.g., biotinylation kit available from Pierce Chemicals, Rockford, Ill.), and immobilized in the wells of streptavidin-coated 96-well plates (Pierce Chemicals).

Alternatively, antibodies reactive with the GPCR142 but which do not interfere with binding of the GPCR142 to ligand component or test compound can be attached to the wells of the plate, and GPCR142 then trapped in the wells by antibody conjugation.

A variety of labels can be used to label either the ligand component or fragments thereof, such as those that provide direct detection (e.g., radioactivity, luminescence, optical or electron density), or indirect detection (e.g., epitope tag such as the FLAG epitope, or enzyme tag such as horseradish peroxidase).

Interaction of the GPCR142 to ligand component in the presence and absence of a candidate compound can be accomplished in any vessel suitable for containing the reactants. Examples of such vessels include microtiter plates, test tubes, and micro-centrifuge tubes.

In another general aspect, the invention relates to a method for identifying a compound that binds GPCR142 and mimics a ligand component, comprising the steps of:
(a) contacting a test compound with an assay reagent. comprising GPCR142 or an active fragment thereof;
(b) measuring a biological activity of the GPCR142 or active fragment thereof; and
(c) comparing the result of step(b) with that of a control wherein the GPCR142 or an active fragment thereof was contacted with the ligand component or an active fragment thereof, in the absence of the test compound.

In one preferred embodiment of this method, the GPCR142 or active fragment thereof is expressed from a recombinant cell, preferably on the cell surface. In another preferred embodiment, the GPCR142 or active fragment thereof is within an isolated cell membrane preparation.

The biological activity can be any of the biological activities associated with the receptor and ligand component complex, or the interaction of GPCR142 and such ligand component, such as the signal transduction event or the changes in intracellular concentration of a second messenger molecule triggered by activated GPCR142. These biological activities can be measured using methods described supra. A test compound that “mimics” the ligand component elicits a similar change in the biological activity of GPCR142 as that of such ligand component.

Further, the receptor can be modified to produce constitutive activity. This constitutive activity can be suppressed by an inverse agonist.

The following examples further illustrate various aspects of the invention and its preferred embodiments.

### EXAMPLES

In the following experimental protocols, human insulin, IGF1, and IGF2 were purchased from Sigma (St Louis, Mo.). Human INSL3, INSL4, INSL6, oxidized relaxin-3 A-chain and B-chain peptides were purchased from Phoenix Pharmaceuticals, Inc. (Belmont, Calif.). Porcine relaxin was purchased from National Hormone & Peptide Program (Torrance, Calif.). Human relaxin3 was produced recombinantly as described by Liu et al., 2003a. Unless otherwise indicated, polymerase chain reactions (PCR) were performed using Expand High Fidelity DNA polymerase (Roche Biosciences) at conditions of: 94° C., 30 sec for denaturing; 65° C., 30 sec for annealing; and 72° C. 3 min for extension for 40 cycles.

All of other peptides were produced recombinantly.

### Example 1

### Molecular Cloning of Different Species of GPCR142

The sequence of human GPCR142 sequence (Genbank Accession No.: AL355388) was identified from the human genome based on its homology to GPCR135. The complete coding region of human GPCR142 was PCR-amplified from human genomic DNA using two primers: forward primer (SEQ ID NO: 10)=5′ ACT GGA ATT CGC CAC CAT GCC CAC ACT CAA TAC TTC TGC C 3′; reverse primer (SEQ ID NO: 11)=5′ ACT GAC GCG GCC GCT CAC CCG GGT GTC CCT CTG TCC AGG T 3′.

The monkey GPCR142 gene containing the complete coding region, the 5′ untranslated region (UTR) and the 3′ UTR was PCR amplified from Macaca Nemestrina monkey genomic DNA using two primers (forward primer (SEQ ID NO: 12): 5′ AGG TGG TGG GTT GTC CTT TCC ACA 3′; reverse primer (SEQ ID NO: 13): 5′ CTC AAG GAT CCT ACA CTT GGT G 3′) designed according to the human GPCR142 gene sequence. PCR was performed as described above, except for an annealing temperature of 55° C. The monkey GPCR142 coding region sequence was obtained by direct sequencing of the PCR products. The monkey GPCR142 coding region was then PCR amplified from monkey genomic DNA using forward primer (SEQ ID NO: 39) (5′ ACT AGA GAA TTC GCC ACC ATG CCC ACA CTC AAT ACT TCT GCC T 3′) and reverse primer (SEQ ID NO: 40) (5′ ACT AGA GCG GCC GCT TAC CCG GGT GTC CCT CCG TCC AGG T 3′) designed according to the newly assembled monkey GPCR142 sequence. The PCR product was cloned into the mammalian expression vector pCIneo between the EcoR1 and Not1 sites. The insert region was sequenced to confirm the sequence identity.

The mouse GPCR142 coding region was PCR amplified from Balb/c mouse genomic DNA using forward primer (SEQ ID NO: 14) 5′ ACG ATA GAA TTC GCC ACC ATG GCC ACA TCC AAT TCT TCT GCC TC 3′ and reverse primer (SEQ ID NO: 15) 5′ ACG ATA GCG GCC GCT CAG ACT TCT CCT GGG GAC ACA GCA G 3′, and the PCR product was cloned into the mammalian expression vector pCIneo between the EcoR1 and Not1 sites. The insert region of the plasmid was sequenced to confirm the sequence identity.

The bovine and porcine GPCR142 genes were PCR amplified from bovine and porcine genomic DNAs, respectively, using two primers (forward primer (SEQ ID NO: 41): 5′ ACC AAT CTC TGA TGC CCT GCG 3′; reverse primer: (SEQ ID NO: 42) 5′ GAG TTG GGG ATC AAA GAT CAG ACT 3′), designed according to the human GPCR142 gene 5′ end and 3′ end UTRs. The PCRs were performed as described above using 55° C. as the annealing temperature. The PCR products were sequenced, and then the coding regions for the bovine and porcine GPCR142 genes were assembled. The bovine GPCR142 complete coding region was then PCR amplified from bovine genomic DNA using two primers (forward primer (SEQ ID NO: 16): 5′ ACT AGA GAA TTC GCC ACC ATG CCC ACG CCC AAC ACC TCT GC 3′; reverse primer (SEQ ID NO: 17): 5′ ACT AGA GCG GCC GCC CAG AAA GAG GAG GGG GTT TAA CTT GC 3′), which were designed according to the newly assembled bovine GPCR142 sequence, and the PCR product was cloned into pCIneo. Similarly, the porcine GPCR142 complete coding region was PCR amplified from porcine genomic DNA using two primers (forward primer (SEQ ID NO: 18): 5′ ATG ATA GAA TTC GCC ACC ATG CCC ACA CCC AAT ACC TCT GC 3′; reverse primer (SEQ ID NO: 19): 5′ ACT AGA GCG GCC GCC CAG AAA GAG GGG GAT TAG CTT GCT C 3′), designed according to the newly assembled porcine GPCR142 sequence, and cloned into pCIneo. The inserts of the resulting clones were sequenced to confirm their identities.

### Example 2

### Expression, Purification, and Iodination of Human relaxin3

The complete coding sequence of human relaxin3 was PCR amplified from human brain cDNA library (Clontech) using the forward primer (SEQ ID NO: 20), 5′ ACG ATC GTC GAC GCC ACC ATG GCC AGG TAC ATG CTG CTG CTG CTC 3′, and the reverse primer (SEQ ID NO: 21), 5′ ACG ATA AAG CTT CTA GCA AAG GCT ACT GAT TTC ACT TTT GC 3′. The PCR product was cloned into a mammalian expression vector pCMV-sport1 (Invitrogen) between Sal1 and BamH1 sites. The cloned cDNA was sequenced to confirm the identity.

The expression vector was transfected into COS-7 cells using LipofectAmine (Invitrogen) according to manufacturer's instructions. Three days after the transfection, the supernatants of the transfected cells were collected, the pH was adjusted to 3.0, and the mixture was loaded onto a Sephadex C-25 cation exchange column. The column was washed with 1 M acetic acid and eluted with 2 M pyridine and 1 M acetic acid. The eluted proteins were loaded on a C-18 BondElut column, washed with 0.1% TFA, and eluted with 60% acetonitrile and 0.1% TFA. The eluted proteins were lyophilized, reconstituted in 50 mM Tris-HCl, pH 7.5, and tested in GTPγS binding assays.

To facilitate the purification of the relaxin3 peptide, an expression vector was constructed that encodes a secreted fusion protein comprising the relaxin3 peptide and a FLAG tag at the N-terminus of the relaxin3. Such an expression vector is constructed by replacing the signal sequence of relaxin3 with an alpha peptide signal sequence followed by a FLAG sequence.

The pro-peptide coding region of human relaxin3 was PCR amplified using the forward primer (SEQ ID NO: 22), 5′ ACG ATA GAA TTC GAT GAC GAC GAT AAG CGG GCA GCG CCT TAC GGG GTC AGG C 3′, and the reverse primer (SEQ ID NO: 23), 5′ ACT ATA GGA TCC CTA GCA AAG GCT ACT GAT TTC ACT TTT GCT AC 3′ with human relaxin3 cDNA as template. The PCR products were cloned into a modified pCMV-sport1 vector, where the poly-cloning sites were modified by replacing the sequence between the Pst1 and EcoR1 sites with a sequence that codes an alpha peptide signal sequence followed by a FLAG tag, 5′ CTG CAG GCC GCC ATG CTG ACC GCA GCG TTG CTG AGC TGT GCC CTG CTG CTG GCA CTG CCT GCC ACG CGA GGA GAC TAC AAG GAC GAC GAT GAC AAG GAA TTC 3′. The human relaxin3 pro-peptide coding region was then cloned downstream of FLAG tag between EcoR1 and BamH1 sites. The resulting clone was sequenced to confirm the identity.

The FLAG-relaxin3 fusion peptide expressing plasmid was transfected into COS-7 cells. Two days after transfection, the recombinant fusion peptide was affinity purified from the cell culture medium using an anti-FLAG affinity gel (Sigma). Briefly, the cultured medium was loaded onto the anti-FLAG affinity column. The column was washed with a phosphate buffered saline (PBS) solution, and eluted with 0.1 M Glycine-HCl, pH 2.8. The eluted protein was neutralized with 1 M Tris-HCl, pH 7.5. The N-terminal FLAG tag was cleaved from the fusion peptide with enterokinase (Novagen), and the wild type relaxin3 peptide was further purified by reverse phase HPLC using a C-18 column and a 0.1% TFA/actetonitrile gradient and tested in a GTPγS binding assay.

To increase the processing efficiency of the pro-relaxin3 into mature relaxin3 by pro-hormone convertase, a furin cleavage site consisting of amino acid sequence RGRR was created in pro-relaxin3 at the junction of the C-chain and A chain. This mutation was created by a two-step overlapping PCR reaction. The 5′ end was PCR amplified using the forward primer (P1) (SEQ ID NO: 24), ACG ATA CTG CAG GCC GCC ATG CTG ACC GCA GCG TTG CTG A 3′, and the reverse primer (P2) (SEQ ID NO: 25), 5′ CAG CCA GGA CAT CTC GTC GGC CCC GAA GAA CCC CAG GGG TTC CTT G 3′, with the FLAG-relaxin3 cDNA as the template. The 3′ end was PCR amplified using the forward primer (P3) (SEQ ID NO: 26), 5′ GGT TCT TCG GGG CCG ACG AGA TGT CCT GGC TGG CCT TTC CAG CAG C 3′, and the reverse primer (P4)(SEQ ID NO: 27), 5′ ACT ATA GGA TCC CTA GCA AAG GCT ACT GAT TTC ACT TTT GCT AC 3′, with the FLAG-relaxin3,cDNA as the template. The 5′ end and the 3′ end PCR products were purified and mixed together as the template for the second step PCR using forward primer (P1) and the reverse primer (P4) as described above. The final PCR product was cloned into pCMV-sport1 between Pst1 and BamH1 sites, and the insert region was sequenced to confirm the identity.

The new relaxin3 expression vector was co-transfected with a human pro-hormone convertase furin expressing vector into COS-7 cells using LipofectAmine as the transfection reagent. The human furin expressing vector was constructed by PCR amplifying human furin cDNA from human cDNA library using two primers with the forward primer (SEQ ID NO: 28), 5′ GAC TAG AAG CTT GCC ACC ATG GAG CTG AGG CCC TGG TTG CTA TG 3′ and the reverse primer (SEQ ID NO: 29), 5′ GAC GAT AGC GGC CGC AGT GGG CTC ATC AGA GGG CGC TCT G 3′. The PCR product was cloned into pcDNA3.1/zeo (Invitrogen) between Hind III and Not1 sites. The insert region of the furin expressing vector was sequenced to confirm its identity. The secreted relaxin3 was then purified using an anti-FLAG affinity column, cleaved with enterokinase, and subjected to reversed phase HPLC as described above. Purified relaxin3 from the transfected cell culture medium was again tested in a GTPγS binding assay. SDS-PAGE showed a single band of the purified protein. HPLC analysis indicated that the relaxin3 pro-hormone was completely processed into mature peptide and uniform in retention time.

The purified relaxin3 was labeled using Chloramine T at presence of Na¹²⁵I (PerkinElmer Life Sciences).

### Example 3

### Expression and Purification of Human Recombinant INSL5

Human INSL5 cDNA was PCR amplified from human colon cDNA (Clontech) and engineered to have two preferred furin protease cleavage sites (a natural site “RWRR” between the B-chain and the C-chain and an artificial site “RWWRR” between the C-chain and the A-chain) by adding a furin cleavage site between the C-chain and the A-chain. This was achieved by a two-step PCR reaction. The first step was performed using human colon cDNA as the template and two primers (forward primer (P5)(SEQ ID NO: 30): 5′ ATA TAG GAA TTC GAC GAC GAC GAC AAG AAG GAG TCT GTG AGA CTC TGT GGG C 3′; reverse primer (P6)(SEQ ID NO: 31): 5′ GTG CAA CAC AAA GTT TGT AAA TCT TGT CTT CGC CAA CGT GAA TGC TTC TTT GAC TTC CAA AGC TC 3′. The PCR reaction was performed at conditions of 94° C. 30 seconds, 65° C. 30 seconds, and 72° C. 1 min for 40 cycles. The resulting PCR product from the first step was used as the template for the second step PCR using two primers (forward primer (P7)(SEQ ID NO: 32): 5′ATA TAG GAA TTC GAC GAC GAC GAC AAG AAG GAG TCT GTG AGA CTC TGT GGG C 3 ; reverse primer (P8)(SEQ ID NO: 33): 5′ ACT AGA AAG CTT TTA GCA AAG AGC ACT CAA ATC AGT CAT GGA ACA GCC ATC AGT GCA ACA CAA AGT TTG TAA ATC TTG TC 3′). The PCR reaction was performed at conditions of 94° C. 30 seconds, 65° C. 30 seconds, and 72° C. 1 min for 20 cycles. The product of the second step PCR reactions was then digested with EcoR1 and Hind III and cloned into the pCMV-signal-FLAG (Liu et al., 2003, Journal of Biological Chemistry 278(50):50754-50764)), a modified version of the mammalian expression vector pCMV-sport1 (Invitrogen) that contains a coding region for a signal peptide for secretion and a N-terminal FLAG tag for purification of the recombinant peptide. The resulting INSL5 DNA expression construct was sequenced to confirm the identity and then co-transfected with furin expression plasmid (Liu et al., 2003a) into COS-7 cells. The recombinant INSL5 peptide secreted into cell culture medium was purified by an anti-FLAG affinity column (Sigma). The affinity-purified peptide was cleaved with enterokinase to remove the FLAG tag. The untagged INSL5 peptide was then further purified by reverse-phase HPLC using a C18 column.

### Example 4

### Radioligand Receptor Binding Assays

Human relaxin3 was labeled with ¹²⁵I and purified by reverse phase HPLC. Cell membranes from COS-7 cells transiently expressing GPCR142 were incubated with ¹²⁵I-relaxin3 either in the presence or absence of various concentrations of different competitors in 96-well plates at a final volume of 200 μL in binding buffer: 50 mM Tris-HCl (pH 7.4), 2 mM EDTA plus 0.5% BSA. The binding mixtures were incubated at room temperature for 1 h and then were filtered through 96-well GFC plates (Packard) and washed with ice-cold washing buffer: 50 mM Tris-HCl, pH 7.4. Fifty microliters of Microscint-40 were added to each well and the plates were counted in a Topcounter NTX (Packard). The radioligand binding results were analyzed using GraphPad PRISM software.

This binding assay was also performed using GPCR135.

### Example 5

### GTPγS Binding Assays

The GPCR142 expression vector described above was transfected into CHO cells using Lipofectamine (Invitrogen) according to the manufacturer's instructions. Two days after transfection, the cells were harvested and the cell membranes were prepared by homogenizing the cells in cold 50 mM Tris-HCl, 5 mM EDTA, pH 7.5 followed by centrifugation at 4° C. 1000 g for 10 min. The supernatant was centrifuged at 4° C. 20,000 g for 30 min and the pellet was re-suspended in GTPγS binding buffer: 50 mM Tris-HCl, pH 7.4, 10 mM MgCl2, 1 mM EDTA, pH 8.0, and 100 mM NaCl using a polytron tissue homogenizer. Protease inhibitors were added to the buffer at concentrations of 1 mM PMSF, 10 μg/ml of pepstatin A, 10 μg/ml of leupeptin. Cell membranes and different concentrations of ligands were added to 96-well plates and incubated at room temperature for 20 min. ³⁵S-GTPγS (NEN) was then added to each well at a final concentration of 200 μM in a final volume of 200 μL. The reactions were allowed to proceed at room temperature for 1 h, and then the mixtures were filtered though a 96-well GFC filter plate (Packard) and washed with cold washing buffer: 50 mM Tris-HCl, pH 7.4, 10 mM MgCl₂. Fifty microliters of Microscint-40 (Packard) was added to each well and the plate was counted on a top counter (TopCount NTX, Packard).

The same assay was performed as described above, except using GPCR135 in place of GPCR142.

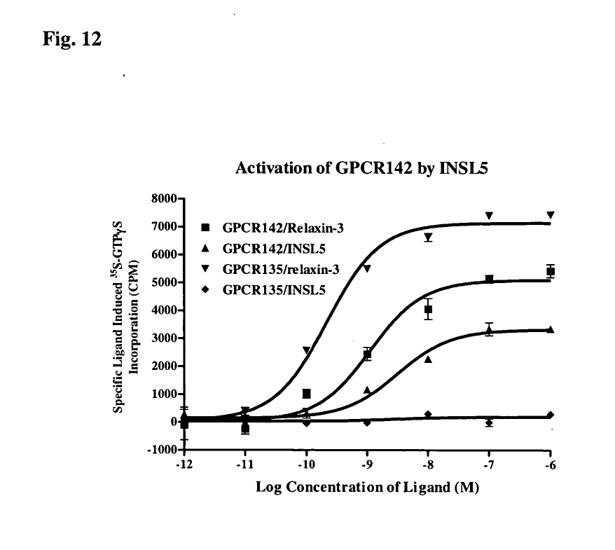

The results of the GTPγS binding assays illustrated in FIG. 3 demonstrate that human recombinant relaxin3 stimulated GTPγS binding in GPCR142 expression membranes and in GPCR135 expression membranes. The results of the GTPγS binding assays illustrated in FIG. 12 demonstrate that whereas human recombinant relaxin3 stimulated GTPγS binding in GPCR142 expression membranes and GPCR135 expression membranes, human recombinant INSL5 stimulated GTPγS binding in GPCR142 expression membranes, but not in GPCR135 expression membranes.

### Example 6

### Intracellular cAMP Accumulation and Measurement

SK-N-MC cells stably expressing GPCR142 were established by transfection of GPCR142 expressing plasmids into SK-N-MC cells harboring a β-galactosidase gene under control of multiple cAMP responsive elements. The stable transfectants were selected by culturing the transfected cells under G418 selection (400 mg/L). The receptor expressing cells were seeded in 96-well plates at a cell density of 30,000 cells/well. After 24 h, cells were stimulated with forskolin or different peptides. Cells were cultured for an additional 6 h and the intracellular β-gal activity, which represents the intracellular cAMP accumulation, was measured as described by Liu et al. (2000), Mol. Pharmacol., 59, 420-426.

The same experiment was performed using GPCR135.

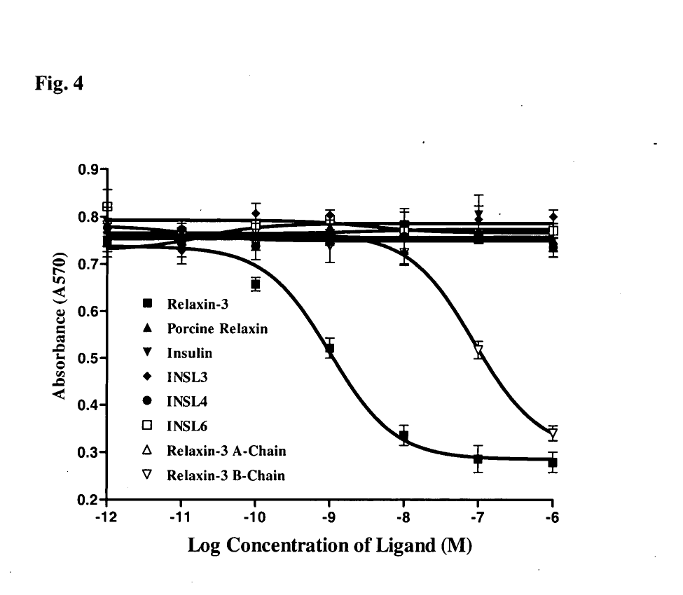

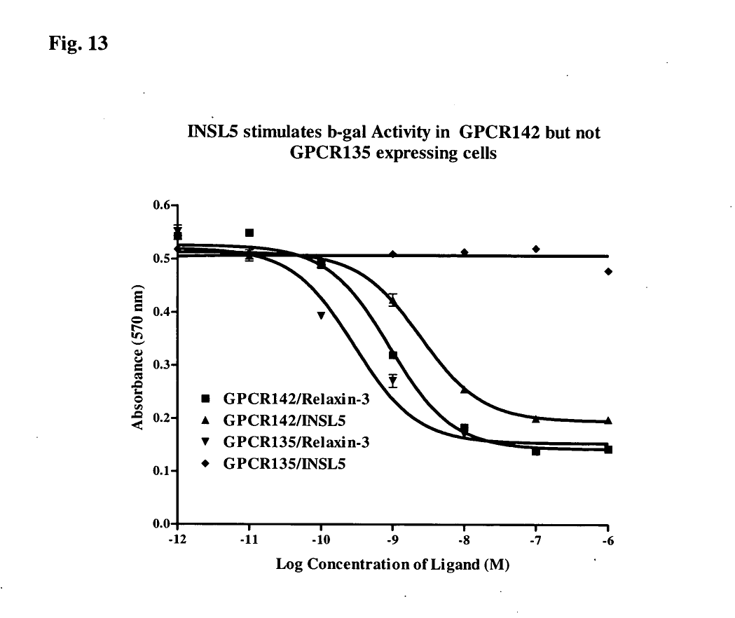

The results depicted in FIG. 4 indicate that relaxin3 dose-dependently inhibits forskolin induced β-galactosidase expression in SK-N-MC cells stably expressing GPCR142. The results depicted in FIG. 13 indicate whereas relaxin3 dose-dependently inhibits forskolin induced β-galactosidase expression in SK-N-MC cells stably expressing GPCR142 and cells stably expressing GPCR135, INSL5 dose-dependently inhibits forskolin induced β-galactosidase expression in SK-N-MC cells stably expressing GPCR142 but not GPCR135.

### Example 7

### Ca²⁺ Mobilization Assays

Human embryonic kidney (HEK) 293 cells were transfected with GPCR135,.GPCR142 or co-transfected with Gqᵢ₅ (Conklin et al. (1993), Nature 363, 274-280) or G_{α16} (Amatruda et al. (1991), Proc. Natl. Acad. Sci. U.S.A. 88, 5587-5591) expressing plasmids using LipofectAmine. Two days after transfection, cells were detached with PBS plus 10 mM EDTA. The detached cells were washed with DMEM-F12 medium (without phenol red, Invitrogen) and seeded in black poly-D lysine coated 96-well plates (BD Biosciences, San Jose, Calif.) at a cell density of 50,000 cell/well. Calcium dye, Fluo-3 (AM) (TEFLABS, Austin, Tex.) was loaded into the cells at a final concentration of 4 μM and ligand-stimulated Ca²⁺ mobilization was monitored using FLIPR (Molecular Devices).

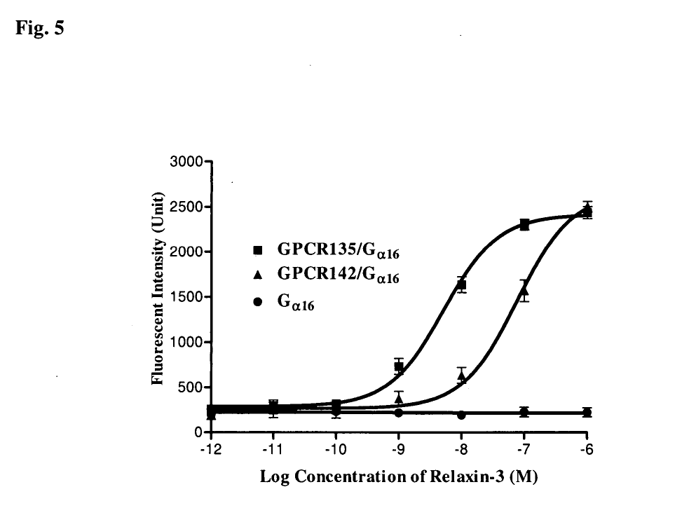

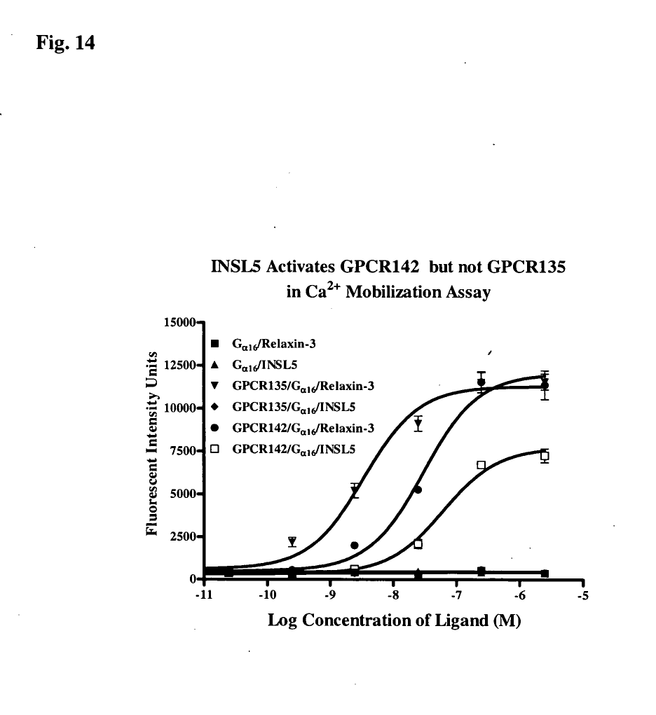

The results depicted in FIG. 5 show that relaxin3 stimulated Ca2+ mobilization in cells co-expressing G_{α16} and either GPCR135 or GPCR142, but not in cells expressing G_{α6} alone. The results depicted in FIG. 14 show that whereas relaxin3 stimulated Ca2+ mobilization in cells co-expressing G_{α16} and either GPCR135 or GPCR142, INSL5 stimulated Ca2+ mobilization in cells co-expressing G_{α16} and GPCR142, but not in cells co-expressing G_{α6} and GPCR135 or in cells expressing G_{α6} alone.

### Example 8

### PCR Detection of GPCR142 mRNA Expression in Different Human Tissues

Forward primer (SEQ ID NO: 34): 5′ TTC ACT GGC CCT TCG GAG GTG CCC T 3′ and reverse primer (SEQ ID NO: 35): 5′ CAG AGA GTG ACC ACA TGG TTG GGA A 3′ for GPCR142 designed according to human GPCR142 coding region were used to PCR amplify human cDNAs from different tissues (Clontech). In a parallel experiment, human β-actin primers with forward primer (SEQ ID NO: 36)(5′ ATA TCG CCG CGC TCG TCG TCG ACA ACG GCT 3′) and reverse primer (SEQ ID NO: 37)(5′ TTT GCG GTG GAC GAT GGA GGG GCC GGA CTC 3′) were used to amplify the human β-actin mRNA as the internal control. The PCR reactions were performed at 94° C. for 40 sec, 60° C. for 40 sec, and 72° C. for 2 min for 35 cycles. The PCR products were run in 2% agarose gels, stained with ethidium bromide and photographed under UV irradiation.

### Example 9

### In situ hybridization

Balb/c mice were perfused with 4% paraformaldehyde and 30 μm thick coronal sections of the whole brain were cut on a sliding microtome and mounted on slides. The tissue was digested with proteinase K, dehydrated and hybridized overnight with GPCR142 antisense or sense probes, at concentrations of about 10⁷ cpm/mL. For post hybridization, the tissue was treated with RNase A, washed, dehydrated and exposed to a Fuji imaging plate for 2 days. The slides were then defatted, dipped in NBT2 nuclear emulsion (Kodak) and developed after 17 days. Arbitrarily, positively labeled cells were defined as any accumulation of silver grains within a cell-sized area that was 3-5 times above background levels.

### Example 10

### cRNA Probes

Labeled antisense and sense probes for mouse GPCR142 were synthesized from cloned cDNA fragments in pBluscript (Stratagene) following linearization with BamHI and HindIII, respectively, and using T7 or T3 RNA polymerase respectively. Probes were labeled with ³⁵S-UTP. The labeled sense strands served as controls and did not show any specific labeling of cellular localization. Specific activities of S-UTP probes were approximately 2-3×10⁶ cpm/μg.

### Example 11

### Radio-Ligand Competition Binding Assays

To characterize INSL5 as the possible ligand for GPCR135 and GPCR142, cell membranes from COS-7 cells transiently expressing human GPCR135 or GPCR142 were used in the radioligand binding assays using [¹²⁵I)-relaxin3 as the radioligand as described by Liu et al. (2003a, 2003b). Briefly, unlabeled relaxin3, INSL5 or other peptides at various concentrations were added to the membranes from COS-7 cells expressing either GPCR135 or GPCR142 as the competitors in the presence of 100 pM of [¹²⁵I]-relaxin3 as the radioligand. The binding assays were performed at room temperature for an incubation time of one hour. The binding mixtures were then filtered through GFC filters pre-saturated with 0.3 polyethylenimine (Sigma) and washed with cold binding buffer. The bound radioligand was counted in a top counter (TopCount/NTX, Packard) with Microscint-40. The results were analyzed by Prism 3.0 software.

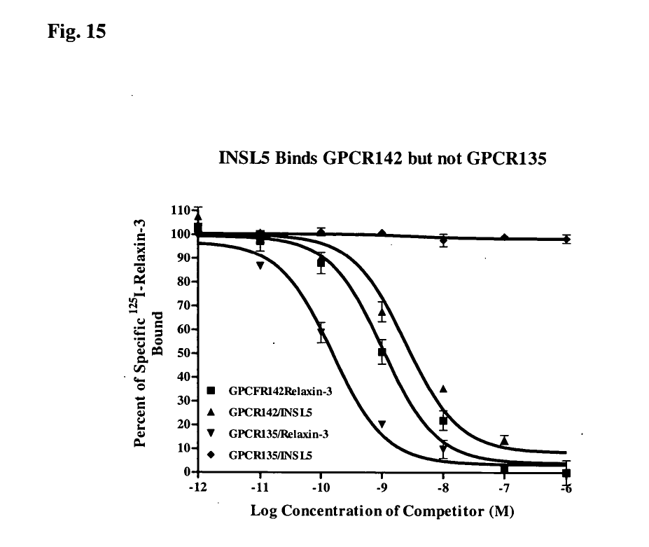

The IC50 values are the concentration of unlabeled ligand that inhibits 50% of the total specific bindings. The results, which are depicted in FIG. 15, show whereas unlabeled relaxin3 specifically displaced the binding of ¹²⁵I-relaxin3 to both to GPCR142 and GPCR135, unlabeled INSL5 specifically displaced the binding of ¹²⁵I-relaxin3 to GPCR142 but not to GPCR135.

Discussion of Various Results

Identification and Sequence Analysis of GPCR142

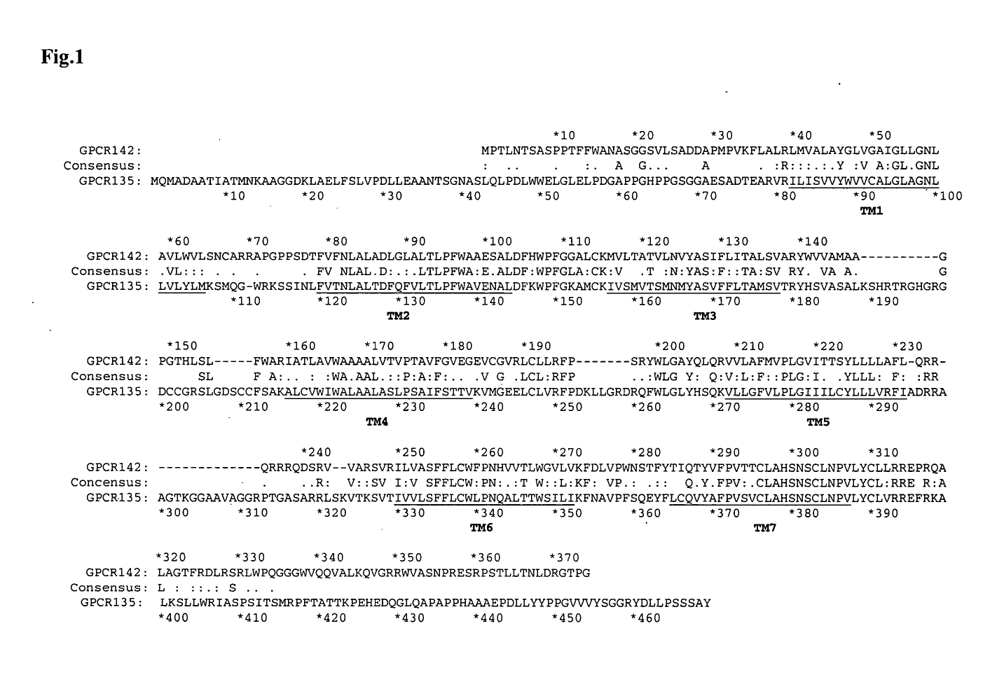

GPCR142 has a 43% sequence identity to GPCR135. At the transmembrane domain regions the two receptors share greater than 50% amino acid sequence identity (FIG. 1). The complete coding region of human GPCR142 was cloned from the human genomic DNA using PCR amplification as described above, and found that the DNA sequence of the cloned DNA confirmed the putative open reading frame of GPCR142. A search of Genbank indicated that the GPCR142 sequence is identical to an orphan GPCR in NCBI patent database (Genbank Accession No. AX148192) and is nearly identical to another Genbank submission (Genbank Accession No. AY288415) except for a single base difference. An open reading frame was found in the mouse genome. Although there is a highly related DNA sequence in rat genome, that sequence does not have an open reading frame to encode a functional receptor for GPCR142.

Characterization of GPCR142 using Radioligand Binding Assays

Using ¹²⁵I-relaxin3 and cell membranes from COS-7 transiently expressing GPCR142, saturation binding and competition binding assays were performed as described above. The results demonstrate that ¹²⁵I-relaxin3 specifically binds to GPCR142 expressing cell membranes. Control cell membranes showed no specific bindings (FIG. 2A). In a saturation isotherm experiment, it was found that ¹²⁵I-relaxin3 bound GPCR142 in a saturable manner with a K_{d} value of 1.9±0.2 nM (FIG. 2B). In competition binding assays it was found that the binding of ¹²⁵I-relaxin3 to GPCR142 is only displaced by unlabeled relaxin3 and relaxin3 B-chain (FIG. 2C), but not by any other insulin/relaxin family members including relaxin, INSL3, INSL4 (Koman et al. (1996), J. Biol. Chem. 271, 20238-20241), INSL6 (Lok et al. (2000), Biol. Reprod. 62,1593-1599), insulin, and relaxin3 A-chain. The Kᵢ values for relaxin3 and relaxin3 B-chain in the competition binding assays were 1.2±0.15 nM and 85±15 nM, respectively.

Functional Characterization of GPCR142 by GTPγS Binding and cAMP Accumulation Inhibition Assays

Results from GTPγS binding assays showed that relaxin3 and relaxin3 B-chain stimulate ³⁵S-GTPγS binding in GPCR142 expressing cell membranes in a ligand-concentration dependent manner. None of the other insulin/relaxin family members stimulated detectable ³⁵S-GTPγS binding to GPCR142 expressing cell membranes. Control cell membranes with no GPCR142 expression did not respond to relaxin3 stimulation in similar assays. The EC₅₀ value observed in GTPγS binding assays was 0.93±0.12 nM for relaxin3 (FIG. 3). Relaxin3 B-chain showed much weaker agonist activity with an EC₅₀ value of approximately 100 nM. Other members of the relaxin/insulin family showed no agonist activity.

GPCR142 was further characterized to see if it is coupled to cAMP inhibition. SK-N-MC cells were used harboring a β-galactosidase (β-gal) gene under control of CRE element as the host cells (SK-N-MC/β-gal). In the cells, the intracellular cAMP concentration is reflected by the β-galactosidase activity. The results showed that relaxin3 dose-dependently inhibits forskolin-stimulated β-gal activity in GPCR142 over-expressing cells with an EC₅₀ value of 0.85±0.09 nM (FIG. 4). Relaxin3 B-chain showed ligand activity, albeit with a significantly lower potency. SK-N-MC/β-gal without GPCR142 expression did not respond to relaxin3 stimulation.

Characterization of GPCR142 by Ca²⁺ Mobilization Assays

Gqᵢ₅ has been reported to be able to shift the signal transduction of GPCRs from cAMP inhibition to calcium mobilization (Liu et al. (2000), Mol. Pharmacol. 59, 420-426). Having found that relaxin3 stimulates Ca²⁺ mobilization in 293 cells co-expressing GPCR135 and Gqᵢ₅, similar experiments were performed with GPCR142. No relaxin3-stimulated Ca²⁺ signaling was observed in 293 cells co-expressing GPCR142 and Gqᵢ₅. G_{α16} has been reported (Amatruda et al. (1991), Proc. Natl. Acad. Sci. U.S.A. 88, 5587-5591) to be able to couple with many GPCRs that normally are not linked to Ca²⁺ signaling and shift their signal transduction into Ca²⁺ signaling (Offermanns et al. (1995), J. Biol. Chem. 270, 15175-15180). When GPCR142 was co-expressed with G_{α16} in 293 cells, relaxin3 stimulated a strong Ca²⁺ signal in the transfected cells in a dose-dependent manner. The EC₅₀ of relaxin3 binding to GPCR142 observed in the Ca²⁺ mobilization assays was approximately 50 nM (FIG. 5). Relaxin3 also stimulated Ca²⁺ mobilization in 293 cells co-expressing GPCR135 and G_{α16} with an EC₅₀ value of approximately 5 nM. HEK293 cells transfected by G_{α16} alone did not respond to relaxin3 stimulation.

GPCR142 mRNA Tissue Expression Profile

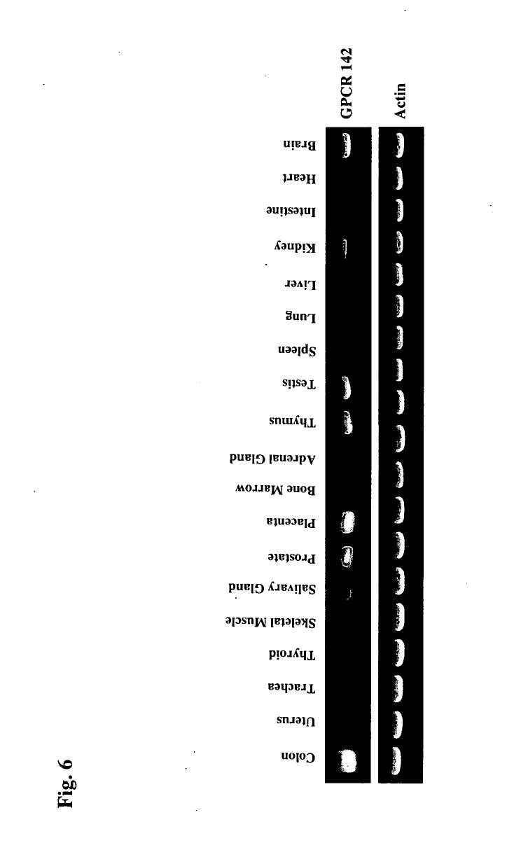

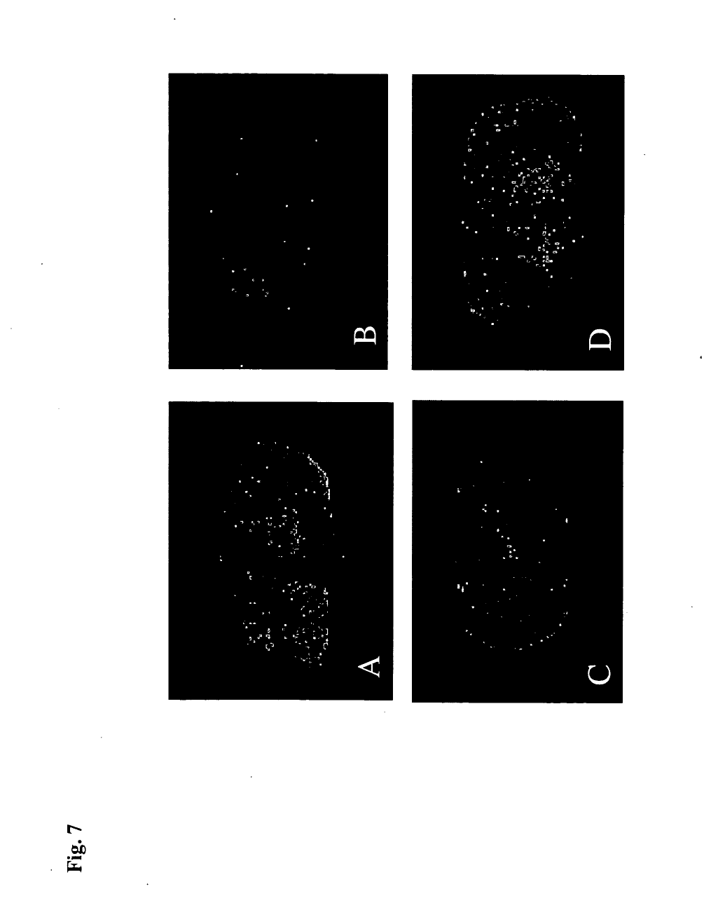

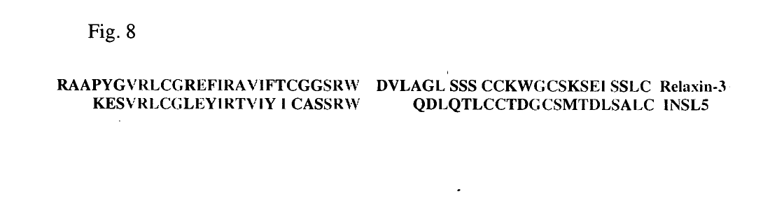

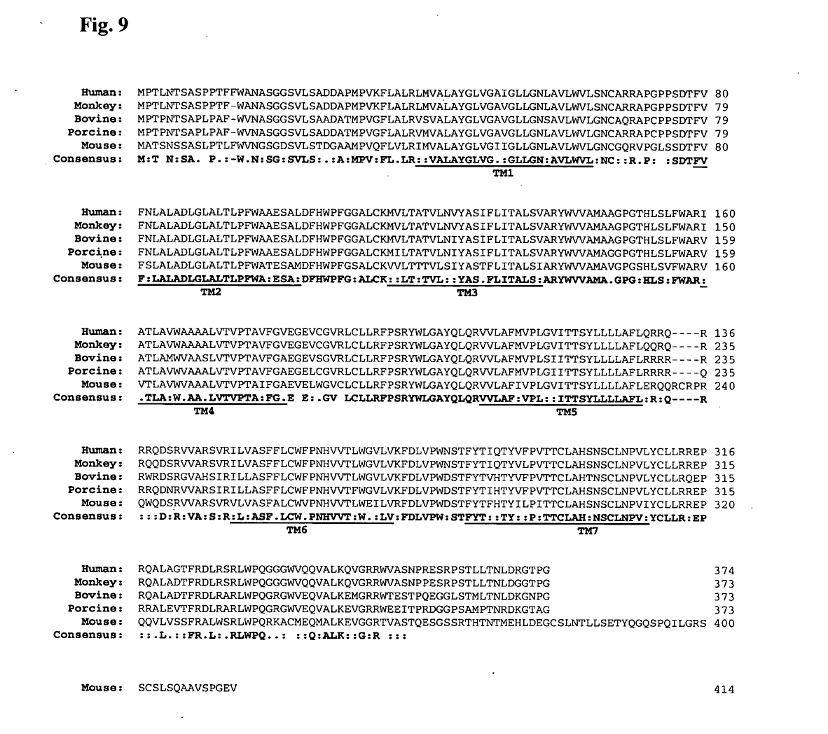

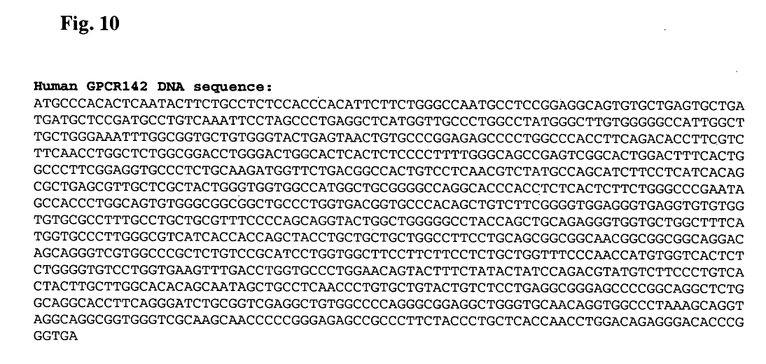

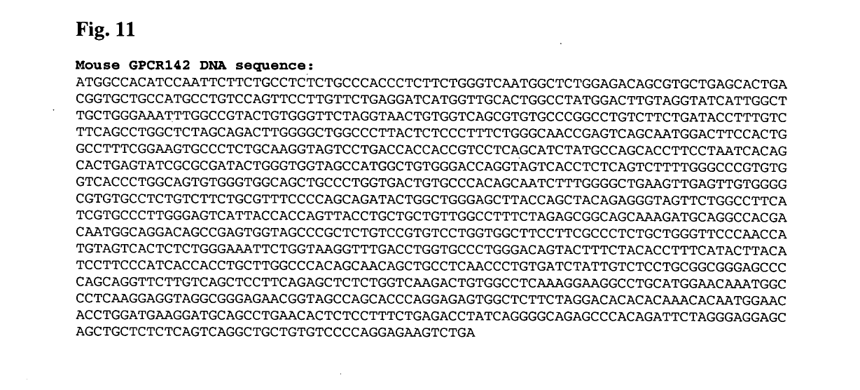

Using RT-PCR, GPCR142 mRNA expression in different human tissues was studied. The results showed that GPCR142 mRNA is detected in many tissues, including brain, kidney, testis, thymus, placenta, prostate, salivary gland, thyroid, and colon (FIG. 6). Using in situ hybridization, the mRNA expression of GPCR142 in the brain was further studied. Because GPCR142 gene appears to be a pseudo-gene in rat, the GPCR142 mRNA brain expression pattern was studied in mouse using a mouse GPCR142 probe. The results showed that light distribution of GPCR142 mRNA is detected in the septal region of the thalamus (FIG. 7).

In summary, GPCR142 behaved similarly overall to GPCR135. In the radioligand binding assays, relaxin3 bound to GPCR142 with a high affinity. Compared with GPCR135, the binding for GPCR142 resulted in a lower signal-to-noise ratio, which may be due to the slightly lower affinity or due to the lower receptor expression levels. In the GTPγS binding assays, GPCR142 transfected CHO cells membranes responded to relaxin3 stimulation. The ligand-stimulated GTPγS incorporation was again consistently lower compared with that from GPCR135 transfected cells, which may be due to receptor expression level or receptor/G-protein coupling efficiency. In cAMP accumulation assays performed in SK-N-MC/CRE-β-gal cells, relaxin3 inhibited forskolin-stimulated cAMP accumulation, suggesting that GPCR142 is linked to Gi/o proteins. The EC₅₀ values derived from cAMP accumulation and GTPγS binding assays agree with the K_{d} and Kᵢ values derived from the bindings assays, which are ranging from 1 to 2 nM.

It was shown that Gqᵢ₅ is capable of shifting the signal transduction of GPCR135 into Ca²⁺ mobilization. One difference between GPCR142 and GPCR135 is that GPCR142 does not couple with Gqᵢ₅. Relaxin3 does not stimulate any detectable Ca²⁺ signal in 293 cells co-expressing GPCR142 and Gqᵢ₅. One reason could be that Gqᵢ₅, an artificial chimeric G-protein, does not couple to all Gᵢ-linked GPCRs. A similar phenomenon was observed when GPR7 and GPR8 was studied (Shimomura et al. (2002), J. Biol. Chem. 277, 35826-35832; Brezillon et al. (2003), J. Biol. Chem. 278, 776-783), two highly related NPW receptors linked to cAMP inhibition. While NPW stimulated Ca²⁺ signal in 293 cells co-expressing GPR7 and Gqᵢ₅, it did not do the same for cells co-expressing GPR8 and Gqᵢ₅. G_{α16} seems be able to couple with both GPCR142 and GPCR135 and shift their signal transduction into Ca²⁺ signaling. However, relaxin3 showed significantly lower potency in Ca²⁺ assays compared with those derived from other assays for both GPCR142 and GPCR135. Similar phenomena for a few other Gᵢ-linked receptors have been observed (Liu et al. (2001), J. Pharmacology and Experimental Therapeutics 299,121-130; Chen et al. (2003), European J. Pharmacology 467, 57-65) and it has been reasoned that the difference could be due to either the non-natural receptor/G-protein coupling or due to the fact that the Ca²⁺ assays are pre-equilibrium assays.

Another difference between GPCR142 and GPCR135 is their mRNA expression patterns. While GPCR135 mRNA is expressed in restricted tissues with the predominant expression in the brain (Matsumoto (2000), Gene 248, 183-189), GPCR142 mRNA is expressed in a broader range of peripheral tissues, suggesting that GPCR142 may have a different physiological role from that of GPCR135.

Although the various aspects of the invention have been illustrated above by reference to examples and preferred embodiments, it will be appreciated that the scope of the invention is defined not by the foregoing description, but by the following claims as properly construed under principles of patent law.

1. A receptor-ligand complex comprising a receptor component containing GPCR142 or an active fragment of GPCR142 bound to a ligand component containing relaxin3 or an active fragment of relaxin3, wherein at least one of the receptor and ligand components is in a substantially pure form.

2. The receptor-ligand complex of claim 1, wherein the receptor component is originated from a human, a mouse, a rat, a monkey, a cow, or a pig, and the ligand component is originated from a human, a mouse, a rat.

3. The receptor-ligand complex of claim 1, wherein said ligand component bears a radioisotope label.

4. The receptor-ligand complex of claim 1, wherein the GPCR142 has an amino acid sequence selected from SEQ ID NO: 2 and SEQ ID NO: 4.

5. The receptor-ligand complex of claim 1, wherein said receptor component is a product of expression on the cell surface of a recombinant GPCR142 host cell.

6. The receptor-ligand complex of claim 1, wherein said receptor component contains the GPCR142 or active fragment of GPCR142 associated with isolated cell membranes or lipid vesicles.

7. The receptor-ligand complex of claim 1, wherein both said receptor component and said ligand component are in a substantially pure form.

8. The receptor-ligand complex of claim 1, wherein said ligand component is in a substantially pure form as a product of recombinant expression.

9. The receptor-ligand complex of claim 1, wherein said at least one of the receptor and ligand components is in a substantially pure form as a product of isolation, peptide synthesis, or recombinant expression.

10. The receptor-ligand complex of claim 1, wherein said ligand component is in a substantially pure form as a product of peptide synthesis.

11. An isolated polynucleotide having a nucleotide sequence encoding an amino acid sequence of SEQ ID NO: 2, or a complement thereof.

12. An isolated polypeptide having an amino acid sequence of SEQ ID NO: 2.

13. A vector consisting of a polynucleotide encoding a polypeptide having an amino acid sequence of SEQ ID NO: 2.

14. A recombinant host cell comprising a vector comprising a polynucleotide encoding a polypeptide having an amino acid sequence of SEQ ID NO: 2.

15. A method of identifying a compound that increases or decreases a biological activity of a GPCR142/relaxin3 complex, comprising the steps of:
(a) contacting a test sample comprising a compound and a buffering solution with an assay reagent comprising a receptor-ligand complex as defined in claim 1;
(b) determining the biological activity of the receptor-ligand complex; and
(c) comparing the result determined in step (b) with a control measurement wherein the receptor-ligand complex has been contacted with the buffering solution.

16. The method of claim 15, wherein the GPCR142 component of the receptor-ligand complex is a product of expression on the cell surface of a recombinant GPCR142 host cell.

17. The method of claim 16, wherein said determining the biological activity of the receptor-ligand complex comprises measuring a second messenger response.

18. The method of claim 17, wherein said second messenger response is measured by intracellular pH, intracellular calcium ion concentration, or intracellular cAMP concentration.

19. The method of claim 16, wherein the assay reagent comprises an isolated membrane preparation containing the GPCR142 or an active fragment thereof.

20. The method of claim 19, wherein said determining the biological activity of the receptor-ligand complex comprises measuring the amount of protein phosphorylation of the isolated membrane preparation.

21. The method of claim 20, wherein the amount of protein phosphorylation of the isolated membrane preparation is measured using a γ-phosphate labeled GTP molecule.

22. The method of claim 21, wherein the γ-phosphate labeled GTP molecule is selected from ³⁵S-GTPγS, ³³P-GTPγP, and ³²P-GTPγP.

23. A method of identifying a compound that binds to GPCR142 or an active fragment thereof, comprising the steps of:
(a) contacting GPCR142 or an active fragment thereof with a test compound and with a labeled relaxin3 or an active fragment thereof;
(b) determining the amount of the labeled relaxin3 or active fragment thereof that binds to the GPCR142 or active fragment thereof; and
(c) comparing the amount determined in step (b) with a control measurement wherein the GPCR142 or active fragment thereof has been contacted with the labeled relaxin3 or active fragment thereof in the absence of test compound.

24. The method of claim 23, wherein the labeled relaxin3 or active fragment thereof is labeled with a radioisotope label.

25. A method for identifying a compound that binds GPCR142 and mimics relaxin3, comprising:
(a) contacting a test compound with an assay reagent comprising GPCR142 or an active fragment thereof;
(b) determining a biological activity of the GPCR142 or active fragment thereof; and
(c) comparing the result determined in step (b) with that of a control measurement wherein the GPCR142 or an active fragment thereof was contacted with relaxin3 or an active fragment thereof in the absence of the test compound.

26. The method of claim 25, wherein the GPCR142 or active fragment thereof is expressed from the surface of a recombinant cell.

27. The method of claim 25, wherein the GPCR142 or active fragment thereof is within an isolated cell membrane preparation.

28. A receptor-ligand complex comprising a receptor component containing GPCR142 or an active fragment of GPCR142 bound to a ligand component containing INSL5 or an active fragment of INSL5, wherein at least one of the receptor and ligand components is in a substantially pure form.

29. The receptor-ligand complex of claim 28, wherein the receptor component is originated from a human, mouse, rat, monkey, cow, or pig and the ligand component is originated from a human, mouse, or rat.

30. The receptor-ligand complex of claim 28, wherein said ligand component bears a radioisotope label.

31. The receptor-ligand complex of claim 28, wherein the GPCR142 has an amino acid sequence selected from SEQ ID NO: 4, SEQ ID NO: 7, SEQ ID NO: 8, SEQ ID NO: 9, and SEQ ID NO: 2.

32. The receptor-ligand complex of claim 28, wherein said receptor component is a product of expression on the cell surface of a recombinant GPCR142 host cell.

33. The receptor-ligand complex of claim 28, wherein said receptor component contains the GPCR142 or active fragment of GPCR142 associated with isolated cell membranes or lipid vesicles.

34. The receptor-ligand complex of claim 28, wherein both said receptor component and said ligand component are in a substantially pure form.

35. The receptor-ligand complex of claim 28, wherein said ligand component is in a substantially pure form as a product of recombinant expression.

36. The receptor-ligand complex of claim 28, wherein said at least one of the receptor and ligand components is in a substantially pure form as a product of isolation, peptide synthesis, or recombinant expression.

37. The receptor-ligand complex of claim 28, wherein said ligand component is in a substantially pure form as a product of peptide synthesis.

38. An isolated polypeptide having an amino acid sequence corresponding to SEQ ID NO: 7, SEQ ID NO: 8, or SEQ ID NO: 9.

39. A vector consisting of a polynucleotide encoding a polypeptide having an amino acid sequence corresponding to SEQ ID NO: 7, SEQ ID NO: 8, or SEQ ID NO: 9.

40. A recombinant host cell comprising a vector comprising a polynucleotide encoding a polypeptide having an amino acid sequence corresponding to SEQ ID NO: 7, SEQ ID NO: 8, or SEQ ID NO: 9.

41. A method of identifying a compound that increases or decreases a biological activity of a GPCR142/INSL5 complex, comprising the steps of:
(a) contacting a test sample comprising a compound and a buffering solution with an assay reagent comprising a receptor-ligand complex as defined in claim 28;
(b) determining the biological activity of the receptor-ligand complex; and
(c) comparing the result determined in step (b) with a control measurement wherein the receptor-ligand complex has been contacted with the buffering solution.

42. The method of claim 41, wherein the GPCR142 component of the receptor-ligand complex is a product of expression on the cell surface of a recombinant GPCR142 host cell.

43. The method of claim 42, wherein said determining the biological activity of the receptor-ligand complex comprises measuring a second messenger response.

44. The method of claim 43, wherein said second messenger response is measured by intracellular pH, intracellular calcium ion concentration, or intracellular cAMP concentration.

45. The method of claim 42, wherein the assay reagent comprises an isolated membrane preparation containing the GPCR142 or an active fragment thereof.

46. The method of claim 45, wherein said determining the biological activity of the receptor-ligand complex comprises measuring the amount of protein phosphorylation of the isolated membrane preparation.

47. The method of claim 46, wherein the amount of protein phosphorylation of the isolated membrane preparation is measured using a γ-phosphate labeled GTP molecule.

48. The method of claim 47, wherein the γ-phosphate labeled GTP molecule is selected from ³⁵S-GTPγS, ³³P-GTPγP, and ³²P-GTPγP.

49. A method of identifying a compound that binds to GPCR142 or an active fragment thereof, comprising the steps of:
(a) contacting GPCR142 or an active fragment thereof with a test compound and with a labeled INSL5 or an active fragment thereof;
(b) determining the amount of the labeled INSL5 or active fragment thereof that binds to the GPCR142 or active fragment thereof; and
(c) comparing the amount determined in step (b) with a control measurement wherein the GPCR142 or active fragment thereof has been contacted with the labeled INSL5 or active fragment thereof in the absence of test compound.

50. The method of claim 49, wherein the labeled INSL5 or active fragment thereof is labeled with a radioisotope.

51. A method for identifying a compound that binds GPCR142 and mimics INSL5, comprising:
(a) contacting a test compound with an assay reagent comprising GPCR142 or an active fragment thereof;
(b) determining a biological activity of the GPCR142 or active fragment thereof; and
(c) comparing the result determined in step (b) with that of a control measurement wherein the GPCR142 or an active fragment thereof was contacted with INSL5 or an active fragment thereof in the absence of the test compound.

52. The method of claim 51, wherein the GPCR142 or active fragment thereof is expressed from the surface of a recombinant cell.

53. The method of claim 51, wherein the GPCR142 or active fragment thereof is within an isolated cell membrane preparation.
